SEQUENCE LISTING (86 sequences)

SEQ ID NO: 1 - DNA, 2244 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 2 - DNA, 2184 bp, Escherichia coli
atggcgcgttccaaaactgctcagccaaaacactcactgcgtaaaatcgcagttgtagtagccacagcggttagcggcatgtctgtttatgcacaggcagcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 3 - DNA, 2124 bp, Escherichia coli
atggcgcgttccaaaactgctcagccaaaacactcactgcgtaaaatcgcagttgtagtagccacagcggttagcggcatgtctgtttatgcacaggcaacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 4 - DNA, 2052 bp, Escherichia coli
atggcgcgttccaaaactgctcagccaaaacactcactgcgtaaaatcgcagttgtagtagccacagcggttagcggcatgtctgtttatgcacaggcatcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 5 - DNA, 1929 bp, Escherichia coli
atggcgcgttccaaaactgctcagccaaaacactcactgcgtaaaatcgcagttgtagtagccacagcggttagcggcatgtctgtttatgcacaggcactgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 6 - DNA, 1764 bp, Escherichia coli
atggcgcgttccaaaactgctcagccaaaacactcactgcgtaaaatcgcagttgtagtagccacagcggttagcggcatgtctgtttatgcacaggcactgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 7 - DNA, 1473 bp, Escherichia coli
tcactgcggcactgtgatggcagagataatttcaaaaccaaaggagcaatcacaatgaaagtgatgaagtggagcgccattgcactggcggtttccgcaggtagcactcagttcgccgtggccgacgcattcgtcagcgatcaggccgaagcgaaggggttcatcgaagacagcagcctcgacctgctgctccgcaactactatttcaaccgtgacggcaagagcggcagcggggaccgcgtcgactggacccaaggcttcctcaccacctatgaatccggcttcacccaaggcactgtgggcttcggcgtcgatgccttcggctacctgggcctgaagctcgacggcacctccgacaagaccggcaccggcaacctgccggtgatgaacgacggcaagccgcgcgatgactacagccgcgccggcggcgccgtgaaggtgcgcatctccaagaccatgctgaagtggggcgagatgcaaccgaccgccccggtcttcgccgctggcggcagccgcctgttcccgcagaccgcgaccggcttccagctgcagagcagcgaattcgaagggctcgacctcgaggcaggccacttcaccgagggcaaggagccgaccaccgtcaaatcgcgtggcgaactctatgccacctacgcaggcgagaccgccaagagcgccgatttcattgggggccgctacgcaatcaccgataacctcagcgcctccctgtacggcgccgaactcgaagacatctatcgccagtattacctgaacagcaactacaccatcccactggcatccgaccaatcgctgggcttcgatttcaacatctaccgcacaaacgatgaaggcaaggccaaggccggcgacatcagcaacaccacttggtccctggcggcagcctacactctggatgcgcacactttcaccttggcctaccagaaggtccatggcgatcagccgtttgattatatcggcttcggccgcaacggctctggcgcaggtggcgactcgattttcctcgccaactctgtccagtactccgacttcaacggccctggcgagaaatcctggcaggctcgctacgacctgaacctagcctcctatggcgttcccggcctgactttcatggtccgctatatcaatggcaaggacatcgatggcaccaagatgtctgacaacaacgtcggctataagaactacggctacggcgaggatggcaagcaccacgaaaccaacctcgaagccaagtacgtggtccagtccggtccggccaaggacctgtcgttccgcatccgccaggcctggcaccgtgccaacgccgaccagggcgaaggcgaccagaacgagttccgcctgatcgtcgactatccgctgtcgatcctgtaatcgaccgacaggcaacgaaaaaacccggcatcgccgggttttttcttcttggcggcaacgcgcctataaaggaagggcgtaggtacc

SEQ ID NO: 8 - DNA, 846 bp, Escherichia coli
atgagtcaaacatcaaccttgaaaggccagtgcattgctgaattcctcggtaccgggttgttgattttcttcggtgtgggttgcgttgcagcactaaaagtcgctggtgcgtcttttggtcagtgggaaatcagtgtcatttggggactgggggtggcaatggccatctacctgaccgcaggggtttccggcgcgcatcttaatcccgctgttaccattgcattgtggctgtttgcctgtttcgacaagcgcaaagttattccttttatcgtttcacaagttgccggcgctttctgtgctgcggctttagtttacgggctttactacaatttatttttcgacttcgagcagactcatcacattgttcgcggcagcgttgaaagtgttgatctggctggcactttctctacttaccctaatcctcatatcaattttgtgcaggctttcgcagttgagatggtgattaccgctattctgatggggctgatcctggcgttaacggacgatggcaacggtgtaccacgcggccctttggctcccttgctgattggtctactgattgcggtcattggcgcatctatgggcccattgacaggttttgccatgaacccagcgcgtgacttcggtccgaaagtctttgcctggctggcgggctggggcaatgtcgcctttaccggcggcagagacattccttacttcctggtgccgcttttcggccctatcgttggcgcgattgtaggtgcatttgcctaccgcaaactgattggtcgccatttgccttgcgatatctgtgttgtggaagaaaaggaaaccacaactccttcagaacaaaaagcttcgctgtaa

SEQ ID NO: 9 - DNA, 1089 bp, Escherichia coli
ttagaactggtaaacgatacccacagcaacggtgtcgtctgaacctacgcccagtttgttgtcagaatcgatctggttgatgatgtagtcaacataggtggacatgtttttgttgaagtagtaggttgcgcccacttcaaagtagttcaccagatcaacatcaccgataccttctacgtctttcgctttagatttggtgtaagcgatggacggacgcagaccgaaatcgaactggtattgcgcaactaacagaacgtcttgcgttttgttggcgaagccgctggtgtttgtaaatttattagtgatcggcgtagcgttacgggtttcaccgtagttcgctgccaggtagatgttgttcgcgtcgtacttcagaccagtagcccactgttcagcttttttaccgttgccaagaggttgagcttcttgcaggttggtacggtcagctgcaccataagcaccaacgataccaaagccttcgtattcgtagctgatagaaccgccaacaccgtcgccgttagaacggcgtgcagtgtcacgctcgtttttacccaggtactgaacagcgaagttcaggccatcaaccagaccaaagaagttggagttacgataggtagcaacgccgccaacacgaccaacgaagaagtcatcgctgtatgcagtatcaccaccaaattctggcagcatatcggtgtaacccagtgcatcataaaccacaccgtagttacggccgtaatcgaaagaaccaacgtcagcgtatttaagacccgcgaatgccagacgcgttttgttaccagtttgagcgtcagcgccttcagagttgttaccctggaagttatattcccactgaccataaccggtcagatcggaattgatttgagtttcccctttaaaaccaagacgggcataggtcatgtcgccattgccaccgtaactgttttcaccgttacccttggaaaaataatgcagaccaacagctttaccgtacagatctactttgttgccatctttgttatagatttctgcagcgtttgcagtacctgctactaacagagcagggacgatcactgccagaatattgcgcttcatcat

SEQ ID NO: 10 - DNA, 1041 bp, Escherichia coli
ttaagcctgcggctgagttacaacgtctttgatacctttaacttcgatctctacgcgacgatccggagccaggcagtcgatcagtgcagcacgctgtttcacgttgtcacaggtgttgccagtaaccgggttggattcgcccataccacgtgcggagatcttgtctgccgggatacctttggagatcaggtaatcaacaacagactgagcacggcgctcggacagaccctggttgtaagcgtcagaaccgatgcggtcggtgtaacccagaacaactacggaaccgtctttcggatccaggttgctcagctggctgtacagctgatccagagcagcctgaccttccggtttcagggttgctttgttgaagttgaacagaacgtcagacttcagagtgaagtgcttggtctgtacttccggtgccggagctggagccggagcaactactggagctgcttcgccctgaccgaaacggtaggaaacacccaggctcagcatgccgttgtccggacgagtgccgatggtgtgtgcgtcaccgatgttgttggtccactggtattccagacgggtagcgatttcaggagtgatcgcgtactcaacaccgccagcgaagaccggagaaacgccggtgtcgtggtttttaccataaacgttggatttagtgtctgcacgccataccatgccacccagacgagtgtagatgtccaggtcgtcagtgattgggtaacccagtttagcggtcagttgaacgccctgagctttgtatgcaccgttttcaacgctgcctttgtacggcatacgacctaaccagtcgtaacccatttcaaagccaacatacgggttaacctggtaaccaccaaaagcaccagcgcccagttggttttcatgggtcgggccattgttgttgatgaaaccagtgtcatggtactgggaccagcccagtttagcaccagtgtaccaggtgttatctttcggagcggcctgcgctacggtagcgaaaccagccagtgccactgcaatcgcgatagctgtctttttcat

SEQ ID NO: 11 - DNA, 906 bp, Escherichia coli
atgaaaaagttattaccctgtaccgcactggtgatgtgtgcgggaatggcctgcgcacaggccgaggaaaggaacgactggcactttaatatcggcgcgatgtacgaaatagaaaacgtcgagggttatggcgaagatatggatgggctggcggagccttcagtctattttaatgccgccaacgggccgtggagaattgctctggcctattatcaggaagggccggtagattatagcgcgggtaaacgtggaacgtggtttgatcgcccggagctggaggtgcattatcagttcctcgaaaacgatgatttcagtttcggcctgaccggcggtttccgtaattatggttatcactacgttgatgaaccgggtaaagacacggcgaatatgcagcgctggaaaatcgcgccagactgggatgtgaaactgactgacgatttacgtttcaacggttggttgtcgatgtataaatttgccaacgatctgaacactaccggttacgctgatacccgtgtcgaaacggaaacaggtctgcaatataccttcaacgaaacggttgccttgcgagtgaactattatctcgagcgcggcttcaatatggacgacagccgcaataacggtgagttttccacgcaagaaattcgcgcctatttgccgctgacgctcggcaaccactcggtgacgccgtatacgcgcattgggctggatcgctggagtaactgggactggcaggatgatattgaacgtgaaggccatgattttaaccgtgtaggtttattttacggttatgatttccagaacggactttccgtttcgctggaatacgcgtttgagtggcaggatcacgacgaaggcgacagtgataaattccattatgcaggtgtcggcgtaaattactcgttctga

SEQ ID NO: 12 - DNA, 1104 bp, Escherichia coli
ttagaactggtaaaccagacccagagctacgatgttatcagtgttgatgccagcgtcacgagtgaactggttgtcgtccagcaggttgattttgtagtcaacgtaggtggacatgtttttgttgaagtagtaggtagcaccaacatcaacatatttcaggatatcttcgtcgtcgtagccacgacccaggtttttacctttagactgcaggtaagccagggacggacgcagaccgaagtcgaactggtactgagcaacagcttcgaagttctgtgctttgttcgcccaacccagggaacctacgcgagttgcgttgtaggtctgggtgtactgagcagccaggtagatgttgttagcgtcgtatttcagaccaccagtgtaggtttcagcacggtcgccgttaccgatgtaagcagcggtgttctgagcatcagtacgtttggagctggagatcgcaccaccgataccgaaaccttcgtaatcataagtgatagaaccgccgacgccgtcgccgttttgacgcagtgcgtcacgaccgttgttagttacgccactagtaaagccttcaccagatgggttgccgtttttaccctggtactgaacagcaaagttcaggccgtcaaccagaccgaagaagtcagtgttacggtaggtcgcgaagccgttaccacgctgctgcatgaagttgtcagaaccgtaggtgtcaccaccgaattctggcagtacgtcggtccaggaagttacgtcataaacaacgccgtagttacgaccgtagtcgaaagaacccacatcctggaatttcagacctgcgaatgccacacgggtccaggagttgttttcgttttcagcgctgttgccctggatctgatattcccactggccgtaaccggtcagctggtcagtaacctgagtttcacctttgaagccaagacgcatgtaggtctggtcgccatctacatctttgttgtcagagaaatagtgcaggccgtctactttaccgtacagatctaatttgttgccgtctttgttgtaaacttcagcagcgtttgctgcgcctgctaccagcagagctgggaccaggagggacagtactttaactttcat

SEQ ID NO: 13 - DNA, 693 bp, Escherichia coli
atgaaaaagattaatgcaataattttattatcttctctgacctctgcctcggtatttgctggcgcatacgtcgaaaacagggaggcatataatcttgcctccgatcagggcgaggttatgctacgtgtgggttataacttcgatatgggcgcgggtattatgttaaccaatacctacaactttcagcgagaagatgaactaaaacatggatataacgaaattgaaggctggtatccgttatttaaaccaaccgataaattaaccatccagcccggtggcttaattaatgataagagtatcggttcgggtggtgcagtgtatctggacgtcaactataaatttgtaccatggtttaatctgacagtacgaaatcgctataaccataataactatagttcaacagatttgagcggggaactggataataatgacacctatgaaattggcacctactggaattttaaaatcaccgataaattttcctatacatttgagccacattacttcatgcgagtgaatgactttaatagtagcaacgggaaagatcatcattgggaaatcactaacaccttccgttaccgtattaatgaacactggctcccttatttcgaattgcgctggttagaccgcaacgtcgaaccgtaccaccgcgagcaaaaccagatccgtatcgggacgaagtatttcttctga

SEQ ID NO: 14 - DNA, 954 bp, Escherichia coli
ttaaaatgtgtacttaagaccagcagtagtgatgaagttatagttttctatacctgctccatttttgctgtagtctgaagtgttattattgtgatcataaagtgaagtattaccttttttattcgtaacccgattccatgcgccttcaacataaacttttgcgttaggtgtgacgtaataacctgcattgactgcaacagaatagtaattttggtctttgaccttactgcgataagtgattctttttcccgggtcatagtgttcatcgttatcagatgattccacccagccgctgtatttaaatgtgccaccgagttcaaaatcttcataacgataacttccagtcaagccaatgtagggcattttaaaacgttgtttgtagccgattgctctttctccattcgggaaggagccgatatcatctctgaatccctcctcagaactgtagatataggaaccacctctggctgtaaagctataacggctttcctgatatccggccatgagtcccaggcggtaattgggttcgttgaggagccagcctttgatattcagatcaaattcgttggcataattgagttgtgtatcagggtgtctactttcatccgtccaggttccggggttactggaatccatccagtcctgatcgaccatattgccacctcggctgccgagagttgtccagccagcagccccgatagatatctggggcatcaaatcccaattaattgcacctttaataattgcagcgttattgaatttccagtcgagttgactgacttttcggcctccttcttcggctagataaacacgctcttttgtttttccgctcagagttccaagactaatgtccgcatttatgttgtcaggagtaaacgataaagtctcggtagaagcaaaagagctgatcgcaataggggttgtcaggactattcccagaagtttcgcccgcat

SEQ ID NO: 15 - DNA, 1056 bp, Escherichia coli
ttaaaactgatacgtcatgccaaccgcgacaatatcatcattattaatattcaatttgttatcgctatccagttggttgattttataatcaacaaacgctgacatatttttgttgaaataatacgtagcaccgacgtcgatataattgaccagatcttcatcaccgataccttcaatatctttccctttcgataagacataacccagcgatggacgcagaccaaagtcaaactggtattgagcgaccgcttcaaagttctgtgtcttattggcaaagccgccagttattggcgtcattttgcgtgtttcagaatagaaagttgccagataaatattattggcatcgtatttcagacctgttgcccatgcttctgcacgcttgcctgtgccacggctttgcaggttctgctcgttggtgcgatctgagttggtataggccccactaatggcgaaatcgctgccgccaaagtcatatgtcaatgacgtgccgaagccatcgccgttttgctttttaacgtcgcggttttcgtttttcccttgatattgcagggttaagttcaggccatcgataacgccgaagaagtcggtgttccgatacgtcgccagaccgctggcgcgtttggtcataaagttgtcggtctgcgccgaggaatcgccaccaaattccgggaacatatcggtccaggcttccacgtcatacaacgcccccaggttacgaccataatcgaaagaacccaaatctttatatttcaacccggcaaaagcgagacgcgttttttgctgtgcagtatcactctctgctttattaccggcaaactctgcttcccaacgaccataaccagtcagttgatcgttaatttgtgtttcgcctttgaaaccaaaacggatataactctggtcgccatctttactggcgttatcactcatataatgcatggctttaactttgccatagacatccagtttattaccgtctttattatatatttctgcagcctgtacagatgcagatgccacaatgcccatcaccactaatgccagagtgctctttttcat

SEQ ID NO: 16 - DNA, 1341 bp, Escherichia coli
atgatgattactctgcgcaaacttcctctggcggttgccgtcgcagcgggcgtaatgtctgctcaggcaatggctgttgatttccacggctatgcacgttccggtattggttggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcacaacagaatgactgggaagctaccgatccggccttccgtgaagcaaacgtgcagggtaaaaacctgatcgaatggctgccaggctccaccatctgggcaggtaagcgcttctaccaacgtcatgacgttcatatgatcgacttctactactgggatatttctggtcctggtgccggtctggaaaacatcgatgttggcttcggtaaactctctctggcagcaacccgctcctctgaagctggtggttcttcctctttcgccagcaacaatatttatgactataccaacgaaaccgcgaacgacgttttcgatgtgcgtttagcgcagatggaaatcaacccgggcggcacattagaactgggtgtcgactacggtcgtgccaacttgcgtgataactatcgtctggttgatggcgcatcgaaagacggctggttattcactgctgaacatactcagagtgtcctgaagggctttaacaagtttgttgttcagtacgctactgactcgatgacctcgcagggtaaagggctgtcgcagggttctggcgttgcatttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgtcggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtcttcgcaacctacgccaagtgggatgagaaatggggttacgactacaccggtaacgctgataacaacgcgaacttcggcaaagccgttcctgctgatttcaacggcggcagcttcggtcgtggcgacagcgacgagtggaccttcggtgcccagatggaaatctggtggtaa

SEQ ID NO: 17 - DNA, 2325 bp, Escherichia coli
tcagaacttcaacgacccctgcatatacagcgtgcgcggctggcctgcatagatgcctttgttgttgtcgtcataagagcggatgaagtagtcctggtcgaagatgtttttcacaccgaatgccaggttcagatctgccatctgcgggccaaagtcatacgccacgcgtgcgccccagagcatgaagccgggaatgcggccggtactgccgtcggcgctctctttcaccgtattggcgttatccgcaaactggctggactggaaatcgctgttcagattgaacgtccagtttcctggcttgtagtccacgcccagcgtgcctttatgtttcggggagaatggtaccagattgccgtaggtgtcgcctttctcgcggatttccgcgttcacatacgcatagctggcgtagatggaaacgttatcaagcgttggcgttagcgtacccagatcgtaacgtgcctgcgtttccagcccggtatggcgcgttttgccacgtgcagtgacggtgtcgttggtctggttggagtcgtactgattgttaaagttaatcaggaacagccccatttccgccgtcagcgcgccgtcgtcgtagcgggtaccgagttcccaggttcgcgctttttccggttcaacattgccgctttgcacagccttgccaatctggctgtactgtacggtgccgaacgagccttcagtgtttgcataaagattccagctgtcagtcaggtgatagagcacgttcaacgccggaagcggtgcgttatagctcacttcttcgtgcgtgcctgtgatggcgttgttctggtatgactcgatatgttcgaaacgcatacccggcgtgatggtccagttgccgatgtcgattttgtcatccagataccaggcgtgcgcctcggtgccggaacgcgtatcgcggtcgtaagggcttgagccggacggcaactgcccgctgctggtggcggtgtagtaacgcatttcatgcgttgattcattcaaatagcgatagcccacgcccacttcgtgcgcggaagggccgatcataaagatctggctgtagcgtggctcaataccgcgcacccagtagttacgcggcgagagggtgatgcgtttgccttgctccaggtagccgctgcgcagggtttgggtgtagaacccctgaatgttgaatttatgctggctgtctggctggaactggtagcccaggctcgccagcttgcgacgaccccagaagcggtcatacgggcgggtggattgccagcgatcggcgtcgtaatccgcgcgagacaggccaccgggcatgtcggcttcaccgtcgtaatattgcagcaggctgttgaaggtgtgcacctcatccggcgcatatttgcttttcagcatcaggtcgtcgatgcgggtggcgctgtgctcgcgccagtcactgccgcgcgtgccggagtagagcagcgcggtgccaaaaccgttgtccgctgtgccgcccaccatcaggttgtgcgtctctttcgggttgttttgtgaagaggttgggctgagctgaccttccacgcccgcctcgataccaaagtcctgcggaatggcacgggtaacaaagttcaccacgccgcccacgctctgcggtccgtaacgcaccgcaccaccaccgcgtaccacgtcaatggcatccatgttgccgagcgaaacgggagccagtgaaagctgcggctgaccgtaaggggcgaaggggacggggatgccgtccatcaggacggtcgagcggctggcgaggcgcgggttcaggccccggatgccaaagttcatcgccaggtcgtggctgccggtgccgttgttttccggcgcgctgacgccagggatgcggttaagtacctcacgcatggtggttgcgccggttttggcgaaatcctcacggcggatcacgtcacgcgcgccagcatgttcaaatacgtcgttttcacgcgcatcacccagccagtcgccgaccacggtcagggcatcttcttttggtgcgggcgcgggctccagcgtccagctgttatttcccagcggttttacctgcagtccgctgccgtccagcagttgttgcaggccgctctcgacgtcgtaatcgccgtgcaggccgttgctctgcttgccgcgcgtcaggctggcgtcaaccgagagggtaaatccgctgtgtgcggcatactgattgagcgctttatcgagcgatcccggtgcgatattaacctgtgcagcaaaagcggaaaacgagagaccggccagcggcagcaggctcaggcgaatggtgttaaccaaaggtgttgttttacgaaaaacgcgtaacggcgtcat

SEQ ID NO: 18 - DNA, 1254 bp, Escherichia coli
ttaagcgacttcattcacctgacgacgcagcagggaaagcgggccggggccgctaagcgtgaacacggaaattaaggtgaagcccagcgccaccagacccagcaccagataagcgccctggaaaccgatgctttcatacatattgcccgccagtacagacataaaaatcatcgccagttgcttaaagaagcagaaacagaccagataaatcgtcgctgaaaaacgcacttcaaactggctggtaatatatttaaagcagcccaccagcaggaacggtacttcaaacatatgcagcgttttcagaataaccacttccagcgctgaggtggcgaacgatgagccaataatacgtacagacataatagtgccagccagcagcagggcgtttttcccaccgatgcgattaatgatcagtggcgcaaagaacataatcgaggcgttaagtaattcgcccattgtcgttacgtagccaaatacccgcgtaccctgttcaccggtagcaaagaacgaagtaaagaaattagcaaactgttggtcaaaaacatcgtaggtgcaggaaacgccaataacatacagtgacaaaaaccacagttttggctgtctgaacagttccagtgccagcttaaggctaaatgccgaatggttggcacctaccgcattggcaaccgtggcagaagagggcgcatccgttttggcgaaaaagagtaaaacggcgaggatgagtgcacagccagagcccagccagaaaacaaactgattattgatggtgaacatgatgccgacaatcgaggcacacagcgcccagccaacacagccaaacatccgcgcgcgaccaaattcgaaattactgcgacggctgactttctcaataaatgcctctactgctggcgcaccggcgttaaaacaaaagcctagataaataccaccaacaatcgatcctactaaaatgttgtattgtaacagtggcccgaagataaaaataaagaacggcgcaaacatcactaacatgccggtaataatccacagcaggtatttgcgcagcccgagtttgtcagaaagcagaccaaacagcggttggaataatagcgagaacagagaaatagcggcaaaaataatacccgtatcacttttgctgatatggttgatgtcatgtagccaaatcgggaaaaacgggaagtaggctcccatgataaaaaagtaaaagaaaaagaataaaccgaacatccaaaagtttgtgttttttaaatagtacat

SEQ ID NO: 19 - DNA, 3150 bp, Escherichia coli
tcaatgatgatcgacagtatggctgtgctcgatatcttcattcttgcggctaaagcggcggcgaaccaccacaaagaataccggaacgaagaagattgccagtaccgttgcggtcaccatcccgcccattacaccggtacctactgcgttctgcgcgccggaaccagcaccagtactgataaccagcggcataacgccgaggataaacgccagcgaggtcatcaggatcggacgtaaacgcatccgcaccgcatcaagcgtcgcttcaatcagacctttaccttctttatccatcaagtctttggcgaattcgacgataaggatcgcgttcttcgccgacaacccaatggttgtgagcaggcctacctggaagtaaacgtcattggtcaggccacggaaggtggcagccagcaacgcaccgataacccccagcggaacgaccagcataacggagaacggaatcgaccagctctcgtacagcgccgccagacacaggaacacgacaatcaacgaaatcgcgtacagtgaaggtgcctggttgccggagagacgttcctgataggacatccccgtccagtcatagccaacaccggtaggcagtttgctcgccagttgttccatcagctccattgcttcaccggtacttttacccggtgccgcctggcctaagatttccatggatggcaggccgttgtaacgttccagacgcggcgaaccgtactcccaacgagaagaggagaacgccgagaatggcaccatctgaccatcagcagcacgaacataccagtcgccgatatcatccggcagcatacggtatttcgcttctgacatgacataaactttcttcacacgaccgcggtcgataaagtcgttcacatagctgccgccccatgcagcgcccagagtggtgttaatgtcgttgatagaaacacccagcgcctgcgctttttcctggtcgatatcaatcttaaactgcggggtatcttccagaccgtttggacgtacgctggtcaacatatcagggtgcttcgctgcttctgcaagcaactggttacgcgcctgagtcagtttttcgtgaccaaggccagcctggtcaatcagctcaaagtcaaagccggttgcagtacccagttccacgattgcgggcaggttaaaggcgaaaaccatcgcatctttgatttgcgagaaagcgcgtgttgcacgcatggtaatcgcttcaactttgttttcttcgcccggacgatcggcccagtccttcaaggaaacgaacgcaataccggtattctgaccacgtcccgcaaagccgaagccgttaacggcgaacaccgactcaacgttgttcttttctttggtcagatagtaatgcgttacctcattgagcactttctgtgtacgttcctgcgttgcacctgctggcagctgaaccatggtcataaacacgccctggtcctcatctggcaagaaggagcttggcagacgcacgaacagataggccatgccgaccacgatgatcagatacagcaccaggtaacgccccgtactgcgcagaataccgcctacgctgtcggtgtagtggtgcgtgctcttctcgaacatgcggttaaaccagccgaagaagccttttttaccttccccgtgatcgcctttggcaatcggtttcagcatggtggcacaaagagctggagtcaggatcaacgccaccagtaccgacagcgccattgctgaaacaatggtaatagagaactgacgatagatagcaccagtagaaccgccaaagaaggccatcggtacgaataccgccgacagtaccatcgcgataccgaccagagcgccctgaatctgccccatcgacttacgggtagcttcttttggcggcaaaccttcttccgccataacacgctcaacgttttctaccacaacgatggcgtcatccaccaacaggccgatggcgagcaccatcccgaacattgttagcgtgtttatcgagaagccaaaggcggcaaggacggcaaaggtcccgagcaataccaccggtacggcaatggtcggaatcaacgtcgcgcggaagttctgcaggaacagatacataaccaggaacacgaggatgatcgcttcgaccagcgttttaaccacttcgtgaatagagattttcacgaacggcgtggtgtcgtatgggtaaacaattttcagacccgacgggaagaacggttccatcttcgccagttcagcacggattgccgcagcggtatccagcgcgtttgcaccggtcgccagcttgatccccagaccggaagccggttggccgttaaactctgcgatgatgtcgtagttctcaccacccagctcaatcttcgcgacgtcacgcagcagcacgcgggaaccatcctgattcactttcagcaggattttgccgaactcttcagtagaggtcagacgcgtctgagcaataatagaggcgttaagctgttggcctttcaccggcggcgtaccaccgagctgacccgccgcaacctgggcgttctgcgctttgatggcggtaatgacatcaaccggcgttagctggaatttgttcagctcattcgggttcatccagatacgcatcgcgtactgtgaaccgaacaactgaacatcacccacgcccgacgtacggctgatggcatctttcatattcgccgccacgtagtcggagatatcctcctgcgtcatggtgccatcggtgttgataacgccgacaaccatcaggaagctgctggatgatttctcaacgctcaccccttgctgctgaacttcttgcggcagcaacggcatcgccagctgcagtttgttctgcacctgaacctgcgcgatatccgcatcagtaccagactcaaaggtcagggtgatctgcacggtacccgtggagtcactgttagaggacatgtacatcaggttatcgataccgttcatattctgttcgataacctgtgtcaccgtgtcctgcactgttttcgcatcagcgccggggtaggaggcggagatcgttactgccggcggtgcaatcgtaggatattgcgccaccggcagtttgaggatcgccagcccccctgccaacatgatgataatggcgatcacccacgcaaaaatcgggcgatcgataaagaaattaggcat

SEQ ID NO: 20 - DNA, 789 bp, Halobacterium sp.
atgttggagttattgccaacagcagtggagggggtatcgcaggcccagatcaccggacgtccggagtggatctggctagcgctcggtacggcgctaatgggactcgggacgctctatttcctcgtgaaagggatgggcgtctcggacccagatgcaaagaaattctacgccatcacgacgctcgtcccagccatcgcgttcacgatgtacctctcgatgctgctggggtatggcctcacaatggtaccgttcggtggggagcagaaccccatctactgggcgcggtacgctgactggctgttcaccacgccgctgttgttgttagacctcgcgttgctcgttgacgcggatcagggaacgatccttgcgctcgtcggtgccgacggcatcatgatcgggaccggcctggtcggcgcactgacgaaggtctactcgtaccgcttcgtgtggtgggcgatcagcaccgcagcgatgctgtacatcctgtacgtgctgttcttcgggttcacctcgaaggccgaaagcatgcgccccgaggtcgcatccacgttcaaagtactgcgtaacgttaccgttgtgttgtggtccgcgtatcccgtcgtgtggctgatcggcagcgaaggtgcgggaatcgtgccgctgaacatcgagacgctgctgttcatggtgcttgacgtgagcgcgaaggtcggcttcgggctcatcctcctgcgcagtcgtgcgatcttcggcgaagccgaagcgccggagccgtccgccggcgacggcgcggccgcgaccagcgactga

SEQ ID NO: 21 - DNA, 1491 bp, Aeromonas salmonicida
ctagaaccaggtttccatctgcacgccaaagttccactcgccaccggcggtgaagttgctctggcccatggcatcgtcgctggcgtagctgtcgagctcggatgaccagtccatccagctggcgaacacccgcagctccggacgctcgaagaagccggcggtctgtgccttgaaggtgggggcgaaggtgagcttggtgaagttgccgttcaccgcctgacgacccttgtagcccttgggatccagatccatgtactgccatgccgcctcatagaccagctcgacgttctggctcagcacctgggccacccgggtattgagggtcagccactggtattcatcccctttcacgtaacgatcctggctctgctcggccagcagagccggtgcgaaacgccagcgcgggttgaggtcggtggcgccaaacacggcgacccgtactgcatcggcatcgggcagcagttcgctgtcggaacccaagcccttgagctcggcacccaggccatgaccgtagaggatcgccgccttggagaagccggggttgatgccaaagaagctgtcggcgtgataagccaccagggtatgggcccccttgtccgccgcctcgcttcccttgccaccgtcgttgatgcgctgttcgttgtcattggccaagaggccgttcaccatccactgccagttgccgaagcggttgttcatggtgaggatatagctctctatgtcggacagaccgctggcactgatgtcgccatagtcgcggccatagagggagaagttcaccttccagctgtcggcaagttgcacgtcatagacaccgccaccggtaccggccaggaagaccacgtccgagtcgagccagtggatgtcgaagttgtcccgatcaaagcgcttgcccgcccacagcaccgcattcttgaaagcgccatcgaagctcgccagattgcctatttcggcaaacacctggcgggtattgaggctcgagtcgctggcggtccagtcgttgctggtctccacgccatctgccagcatcagcttgtagcgtgcccagctgccgtcatcaaacgtctgggtcttgagcagattcgcttccatgtaggtgtcgtcttcgttgccaagccggcccactgcgccccccacagatcctgccggcgtgatgtagggaccgccccgcccaccgttgccattgccgttgaccaggaggccagagcgggcatagccgtgaaactcgaagccctgtgcccccgccgtcttctcatccaccttctggctgcgggtgttggcgacctgggcctcctcactggccgcactcgcctttatctcagcctgactggcacgactctccgctgcctcggctcttgcttcggccgcctgtacccgcgcctccagcgccgccagcctggcttcgataccgccctccgccgcccacagcgggctgctcatccccaatcccagcgccactgccaacgccaatttgctcttgaacatggtcat

SEQ ID NO: 22 - DNA, 2241 bp, Escherichia coli
tcagaagtgggtgtttacgctcatataccacgtacgtcccggctcgttataggtatacgccccggcaccggcgatatagttggcccctgccaaatcgcccgtggtctgggcattacccgcacgccacaaacgtttgtcgaacagattgtccacgccgccggtcagactgacattcttcgtcacatcccaggtcgcgctcaggccaacaatgctgtaaggactaatttctttggtttccggtccaaccgctggctgacctttatagttgtacttcttcggctgctgcttgccgtaccaggtgaaggtcgtttgcatcgacaaatcttcccgtgcctgccagctcagcgttgagttcaacgtatactccgggatgatcgacaaacggtcgcccgtggttttgttttcactcttcagcatataagtgatgttattggtccacatcaccgtttcgctaaccggtacgtttaacgatccttccagaccttcaaccaccgctttcggcacgttatcccactgatagagatcggtgccgactgcgttttgccctacagccacatagcccgcttcaatcttattgcgataatcgttacggaaccaggtgacgcccgccagccacccgtcgcgtttgaactccagaccaatctctttgttgatgctggtttctgctttcaggtcatcgttaccttgcagatagcagccgcccgcgctggcatagcaaccctgacctttactgtagagaatgtagttcgggttagtctggtacaggctcggcgctttataagcacgggcgatgcccattttcagcgtgaagtcatcgcctaaaccttgcgatatgttcagcgccgggctccagttattgccgacaatactgtgatgatcgaaacgcagccccggcgttacgatggtgctgtcagtcagctccatgttgttttcggcaaacagcgagaaaatttctgcttttgaatacgggctacggtcggtggtactcacgccatcaatagcgccaccggtattcgttccggtcagtgcctgggtgttggaacttaagtccttcatccgttgctgattccactccgtacccagcgtcagcgtctggttaacgaggaaatcaatcggcaggttaacttcgctgtgcagcatcacgtcatcaagatcgatatcgacgaaatcctgtgtcgctttttcgttaaatttcccttcggtaccgcccgccagaccttccggaatacgcgagttacgggtgtgttcgtactgcacccagttgctggtggtcacgccgttatcccagccaccgttccaggtcagcgcgtagttctggcgatacagacggttggtttcatcgccatatttcgagcgggtataggaatcggagttggtattctgggtgtcgcccgcatacaggttaccctggcggctgtaacctgcttccagttccagcgattgcaatggcgcgaaatcccagcgcaccacgccattaatatctttgttgattaccccttcgcgcccggctggtaacgtcgtggcatacgttccggcacgcgcggactgatggccctggttgatatcccacgcgtcagcctgggttttgtcgaggttgccatacaaacggaagctgaattcgtcgcccagcggaccggtcaggctaaagttagtgcgtttggtggcaccttcctctttatgttctggcgcattgaaatatgcgtcccaggagccgtgccactcgccgctgccttttttggtaatgatgttaaccacgccgcccgccgcgccgttgccataacgcgcagctgccggaccacgcagaacttcaatacgttcaatcatttcaggtggcacccaggaagtatcaccacgggtatcgcgctcgccacgccagccctgacgcaccgagttacggctgcttaccggcttgccgtcaatcaaaatcagcgtgttttccggacccataccgcgaatatcaatctgtcggttattcccacgctgaccactggtggagttaccggtcaggttaacgcctggcatggtacggatgatcttcgacacatcgcgggcaaccgggtttttgcggatttcatctgcggtgatggtcgaaacgccaggcgcctgtaagttctgctcggcggcggtaacgacaatagtatcgtcatgtgaaacaggagtatcggtcggctcttgtgcctgcgctaccccataaatccccagattgaccaacaaggccagggaatgaatcttcttgttcat

SEQ ID NO: 23 - DNA, 2190 bp, Escherichia coli
tcagaattgatacgtgccggtaatgctgaaattacgcggtgtgccgtagacgatagaaccttccacgttggtatcgtaggttttgtcgaacaggttattgacgttcccctgtaacgagaagtttttcgtcacctggtagcgggtgaagagatccaccagcgcgtagctaccttgctcggcgcggaaggtgccatacggcgtcacggtgtcggtatacacgcgattttgccagttaacaccaccgccgaccgtcaactctggcatgacaggcaaccgatagctggtgaacattttaaccgtggtgcgtggcagattaggattaacggcgtttccttcgttatcctctgcaatatagcgcgttgcgccaaatgtcagctgccagttgtcggtaattgcgccgttgagttcaaattccacccctttactgactgtcccatccaccgctttataggcggtttcgccgttgctgccggggataggtgtaccggtggactgagcgacattatcctgctcaatacggaagatggctaacgtggtggtcagacggctattcatccagtccgatttcagacccagctcgtagttgttaccggtgattggagccagatatttgcctgaactgtcacgatcattttgcggctggaaaatagaggtatagctggcgtaggtcgaccagttgtcattgatgtcaaacaccagaccagcgtaaggcgtggtgtggtttttctccatgctgtaagtcagcgtatcaacccgccagttggtataacgtgcgccgaggatcagatgcagcggatcggcaagggtgacacgagtggcagcatataacgatttcatatgtgtggtatcgtcctgcgccaggctctgtggtgaccagtcggtttgtgggaaattgccattaaagttgtagaaactgccaatttcatccgggaagatgttggcccatgaactgaagtaacgattgttttgtttgctgtaactgccaccaaacattagattgtgctgacgaccaaacaattcataactaccgtcagcgaacaaatccagcgcatcaactttacgtttgccactgttccaaccggtgccgccgacataatcaaagccaggtccataattactgtatggcccaaccagcataccatccgctttgtttacataggcatcgacatacatcattttgctgtcaaattcgacttcagagtgggtggcattcagtgtcgcttgccaggtatcagcaaactgctgcttcagggtcataaagaccttgttgatctctttatcgttgtacgcccagtcaggtgcggtactgcgtgcgcgatcgtaactgttgctgctgccatcagtattccagcgcggtaaaccgccccaggtagggctattaacatcaatgcgctgatattcgtaaccggctgaaagcgtcgtaagatcgcctaaatcagcatcgacaatgcccgagaagaaggtcttttcactgttgtagcggtccagccatgagtcgttattctggtagccgccgacaattcgcgcgcggattttaccgtcttcggtgagtgggctttgtaaatccgccacataccgttctttgttccagctaccgtattccgccgagacatcgcctttaaattcacgactggtcgcgtgttttcgaaccatattaattgccgcagatggattacccgtcccggtcatgagtcctgtcgcgccacgcactacttctacgcgttcaaagagtgccatatcagaaagtgcgtcgcccagattccagcgcgattcaaaataggtggggataccatcaaccatatagttatcgatctggaatccgcgggaataataaagagcacgatcggaatccgcctgacttttgctgatccccagcgtgttttccatcacttcgcccagcgtttgtaactgctgatcttccatccgctgctggctaacaatagtgaccgactgaggaatatcacgttgagtcatctgcattttggtacccgcagaggtagacgttacgctgtaatcattttcgccatcatctggagctgtggctgaaccctcaacaatcaccgtttcttcagtggctggtgcagcaaaagcggcagaaggtaatagtgccagtgctatgcaaccggcaagtagtgacggtttggtgatggcttgatattgattatccctgttaaattgtgttgaaagcat

SEQ ID NO: 24 - DNA, 1359 bp, Escherichia coli
ttaatatgcgcgcagcgtctggaaaaccgaggccaaaagacgtgcgtctttgatgcccggttgcgactctacagcagaattaaaatcaagtccggcgcagccggtttgtgccgcttccacgcagttatctgcgcctaagccccccgccagcagaacgttgccaagcgattgaccatttaatagtgaccagtcaaaacgttgcccgcttccaccctggccgttgtctaaaacatatttatcaacgtgctgaaactcgcgggcgggcagggtttcaccgacgcttaatgctttccagatggcaacatgtgctggcagagcttcacgcagcgtatcgatatacagctgttcttcattaccatgcagttgcactgccgccagcgataacaccttagctttgtccaccacatcggcaatatcgtgattgcggaacacgccaacatactgcaacggtgccgcagccatcacttcctgcgcctgttcaacgttgacgcaacgcggtgatgtcgcaacaaaaatcaacccaccgtaaatcgcgcccgcgtcataagctgctttagcatcttgcccacgcgtcaggccacatactttattctcacccagcaacacccggcgcacggcggcgtgcaaatcgtcatgggccatcaacgccgaaccaatcagaaaaccgttagcgaagtggcttaactcgcgcacctgagcgtaagtattgatgccggattcgctgattaccgtcacgttgtgccccagtttcggcgcaagctcgcgggtacggttgagatcaatcgacaaatcacgcagatcgcggttgttgatgccaacgacctttgctcccaatgcaatggcgcgctcctgttcctcttcattactgacttcggtcagcacccccatctccagactgtgagcgacggcggcaagctggcgatattggtcgtcatccagtactgaaagcattaataagcaggcatcggcctggtaatagcgcgccagatagatctggtaagggtcgataatgaagtctttacataaaatcggctgcggggcgatttggctgacgatggggaggaaattaaagctcccctgaaaatatttctcatcagtcagcaccgaaattgccgaagcgtaatgtttataaatggcggcaatgcgtgctggatcgaaatcatcacggatcacgccttttgacggcgacgctttcttgcactccagaataaacgccgtgcgcgcaccctgtagcgcatcataaaaatgtcgcgtgctcggctgaacctcattctgaaaactggccagcggttgctgctgtttgcgggcttctacccaaatcgccttgtctgcgacgattttcgctaaaacggtttgcat

SEQ ID NO: 25 - DNA, 870 bp, Escherichia coli
tcaaaacaaatcgtttagcataacccccacaccgacacgggtctggttgaagttatagtcgatgagcgattcgccatagccgctgtaaacctgagtataaaggcgcacatgtttggtgatcgggtaacttaagcctaactccgcgccgccgtagccggtgttccagttgtactgtcctttcgcactgagcaccgcatcaccgaggtgatagccgattttaagctggtagtaacccatatatttggtgatatccgggttatcgtcagtattacccaccacataccacggcttcacttctaccagccagttaccgttttctgccatcaggcgagtataaaggcggttccagctgcgggaggtcgggtcggaacgcccgttagagtcgtggttataccccatctccacatcgcgcagcgtccaacctgcaaaacggtaatcggtggcaaaaccgaggaacaattgcggttcgtagttggtttcacgaaacggtgaagactcttcgctattggacagttgccaccaggatttttgcgtataagacgcacccaacaccgagttcgggcctaaaatcccacgccacagcggaaatgccaggctcaactgaaactttacttcatccttacgcgcattttccgcccagtcgtaactggcaatcgcttctttattcagatcgctggtttgggtgtaaatgaggtagttggtgtcataaggatagagcgtgaacggattgtcatgctcctgcagcatattggcgataatactgccacgcaccgctggcgcgtcatgcacctctttcaccgttgcctcttgtgcatatactgccataggcaacataaacaccggcaacaaccagccctgcagagtccgcat

SEQ ID NO: 26 - DNA, 1992 bp, Escherichia coli
tcagaagcgataatccactgccataaagtaacgacgtccgtcttcgttatagctgtagtcgtcacgactgagatccttgtcgccaaggttcagcacgcctgcgcgcagtttgacgtctttagtcacctgccaggccgcgccggtattccagatggtataaccgcccggtgttttagccgtcgcgctgtcggcgcgtttctgcccggtatagtgcccagaaacatagaatgaccagtcttccagcgccagcggtttccagtccagcgtaccgttagcagtatggaacggcagatcggatagcggtttgttttcgccgttgctgacatcacgaccatcgttgtaggtgtagttgatcgacagtttccattcatcgttgaacggaattttcagttcggtttccacgccctgaatacgagctttgttaacgttgtagtagctaaataccggtatgcgccgtccgttagcgcccgtctcaaaaccaacaaagttttggtagcccggtgcagcgttgacgtcagacgtacggctgatgctgatacgatctttcacatcgttacggaaaacggtaacgctggattcaaccccttccagccagccttcttcacccatgtagtaaagccccagctcccaactttcgctggtttctggtttcagatccgggctacccacaatcttacatgcgccacggcaggaattgctcgtccagtcagggctaagttgcaacagagaaggtgctttaaatgccgtcgcccagccccctttcaccgttacggtgtcggtggcgttataaaccaggtaggcacgcggactccagtgttcaccgtaggtttcgtgatcgtccatacgcacgccggtcgtcagcgccagcggctcgaagatccgccattcatcttccacaaacagcgcgtactggctggcagacgttttggagctggttcccccggtcaggttcaccgcatcgctaagtttgtcgtgacgccattcaccgccaaccgtgagaaactgattaatcgccgtcagcggcaacgtgtatttgccgtcgaccgtattgctttcggaagttatcgggctgctgttgccagggtttttgttctcgactttctcaccgtagtatttcagttcgctggtgccgtaatcccaacgcccattatggctgacggagtagttctggcgttccaggcggtttttgtccagcgagtcggaatcacgatcctgacggtcgaaaccgtatccggcagtaaaatcgtgattttgattcggtgtccaggcaaattcgacattgccgtcgcggctggagaatccttcaatacgcggcgtttctccggtatcggtggtcgttgagttttgcgggtcatccttttcacgttttgccaggctgccgtaagctttcattcccagcacaccatcaattaatggtccactggtaaagaactgaccgttataggtgtcaccgcgatcgcgatgttcctgaatggtggtatcgacggtaacggtacccgaccatttctgaccgatttttttggtgatgatattcactacaccgccgagcgcatcggaaccgtacagcgacgacatcgggccacggaccacttcaatacgttcgatggaatcgaccgggatccagttcagatcgaaatcattgtggcggaagacggcattgcgggagttcacgcgtttaccgtcgacgagaatcagggtatagctgctgtccagaccacgaatactaacgcccttacggttatccccttcgttcgtcagttgtacgccaggcacttctttgaggacatccttcagattctgtaccggttttcgctgcaggtcttcctgggtaatgacgctgatactggcaggtgcatctttaagattttgttccacggaagatgcagtgacaaccatcgtttcgccatcatcatcgaccgctaacacaggccatgcacaagaaatagcggacaaacacagcccgacccgtacgaaagggttcaacctaaacat

SEQ ID NO: 27 - DNA, 3363 bp, Escherichia coli
atgactatgttccagtattacaaacgatcacggcattttgttttttcagcatttattgcttttgtttttgtcttgttatgccagaacacggcgtttgcgcgggcgtcatcgaatggtgatctgccgacaaaagcggacctgcaggcgcaacttgactcactaaataaacaaaaagatctttctgctcaggacaaactggtgcagcaggatctgacagatacattagccaccctcgataaaatcgatcgcataaaagaagagacagttcagctacggcaaaaagtcgctgaagcgccggaaaaaatgcgccaggcgaccgcggcgttaacagcacttagcgatgtcgataacgacgaagaaacgcgcaaaattctgagcacgctgtcgttgcgccagctggaaactcgcgttgcccaggcgctggacgatttgcaaaacgcacaaaacgatctggcgtcttataacagccagctggtttcgttacagacgcagcccgaacgcgtgcaaaatgcgatgtataacgcttcgcagcagctgcaacaaattcgcagtcgtctggatgggactgatgtcggcgagacagccttacgtcccagccagaaagtgttaatgcaggcccagcaggcgttgctgaatgcggagattgaccagcagcgtaaaagcctggaagggaacaccgtcttgcaggataccttgcaaaagcaacgtgattacgtgacggcgaacagcgctcgtctggagcaccagttacaactgttgcaagaagcggtaaacagcaagcgcctgactttaaccgaaaaaacggcgcaggaagccgtctccccggatgaagccgcgcgtattcaggctaatccgctggtgaagcaggaactggaaattaaccagcagttaagtcagcgtctgattaccgcgactgaaaacggtaatcagttgatgcagcaaaacattaaagtcaaaaactggctggagcgggcgctgcaatcggaacgcaatattaaagagcagattgccgtcctgaagggcagcctgctgttgtctcgtatcctttaccagcaacaacaaacgctgccctcggcggatgaactggaaaacatgaccaaccgcatcgcggatttgcgtctcgaacagtttgaagttaaccagcagcgtgatgcactcttccagagcgatgcgttcgtcaacaaactggaagaaggtcacaccaacgaagtcaacagcgaagttcacgatgcgttattgcaagtggttgatatgcgtcgcgaattgctggatcaactcaacaaacagttgggtaaccagctgatgatggccattaacctgcaaatcaaccagcagcagttaatgagtgtgtcgaaaaacctgaaatccatcctgactcagcaaatcttttgggtgaacagtaaccgtccaatggactgggactggatcaaagcgttcccgcaaagcctgaaagatgaatttaagtcgatgaaaatcacggtgaactggcaaaaagcctggcccgccgtttttatcgctttcctcgctggtttgccgctgctgttgattgccgggctgatccactggcgtctgggctggctgaaagcgtatcaacaaaaactggcttccgctgtgggttccctgcgtaacgacagccagctcaacacaccaaaagcgatccttatcgacctgatccgtgcgctgccggtgtgcctgattattctcgcggttggcctgattctgttgaccatgcagctcaacatcagcgaactgctatggtcgttcagcaaaaaactggcgatattctggctggtgtttggcctgtgctggaaggtactggagaaaaacggcgttgccgtacgtcacttcggcatgccggaacagcagaccagccactggcgtcggcaaattgtccgcatcagtctcgcattgctgcctatccatttctggtctgtggtggcagaactttccccgctgcatctgatggatgatgtgctggggcaagcgatgattttcttcaacctgctgctgattgccttcctggtatggccgatgtgccgcgaaagctggcgtgataaagagtcgcacaccatgcgactggtcaccattaccgtgctgtcgataatcccgattgcgctgatggtgctgactgctacaggctacttctacactacgctgcgtctggcaggacgctggattgaaaccgtttatctggtgatcatctggaacctgctgtaccagacggtactgcgtggcttaagcgtagcggcgcggcgtatcgcctggcgtcgtgcgctggcgcgtcggcagaatctggtgaaagagggcgcagaaggtgctgaaccgccggaagaacccaccattgcactggagcaagttaaccagcagacgctgcgtattaccatgttgctgatgtttgcgctgttcggtgtcatgttctgggcaatttggtccgatttgatcaccgtgttcagctatctcgacagcatcacgctctggcattacaacggcactgaagctggcgctgcggtggtgaaaaacgtcaccatgggcagtctgttgtttgcgattatcgcctcaatggtggcctgggcgttgattcgcaacctgcctggtttactggaagtgctggtgctctcgcgactgaatatgcgccagggcgcgtcgtatgccattactaccatccttaactacatcattattgctgttggtgcgatgacggtgttcggatcgctgggcgtctcttgggataaactccagtggctggccgcagcattatccgtaggtcttggttttggtttacaagaaattttcggtaacttcgtctccggtttgatcattctattcgaacgtccggtgcgtattggcgatacggtaaccattggtagcttctcggggacggtaagtaagatccgtattcgtgcgacaacgattaccgatttcgatcgcaaagaagtgatcatcccgaacaaagcgtttgttaccgagcgtctgatcaactggtcgttgactgacactactacgcgtctggtgatccgtctcggcgtggcctatggctccgatctggaaaaagtgcgtaaagtgttactgaaggcggcgactgagcacccaagggtgatgcacgaaccaatgccggaagtcttctttacggcatttggtgccagcacgttggatcatgagctgcgtctgtatgtgcgtgaactgcgtgaccgtagtcgtactgtcgatgagctgaaccgtactatcgatcagctgtgccgtgaaaacgacatcaacattgcctttaaccagcttgaagtgcatctgcacaacgagaagggcgatgaggtgacggaagtaaaacgcgactacaaaggcgatgacccgacgccagcggtagggtaa

SEQ ID NO: 28 - DNA, 1347 bp, Escherichia coli
atggtcatgagccagaaaaccctgtttacaaagtctgctctcgcagtcgcagtggcacttatctccacccaggcctggtcggcaggctttcagttaaacgaattttcttcctctggcctgggccgggcttattcaggggaaggcgcaattgccgatgatgcaggtaacgtcagccgtaaccccgcattgattactatgtttgaccgcccgacattttctgcgggtgcggtttatattgacccggatgtaaatatcagcggaacgtctccatctggtcgtagcctgaaagccgataacatcgcgcctacggcatgggttccgaacatgcactttgttgcaccgattaacgaccaatttggttggggcgcttctattacctctaactatggtctggctacagagtttaacgatacttatgcaggcggctctgtcgggggtacaaccgaccttgaaaccatgaacctgaacttaagcggtgcgtatcgcttaaataatgcatggagctttggtcttggtttcaacgccgtctacgctcgcgcgaaaattgaacgtttcgcaggcgatctggggcagttggttgctggccaaattatgcaatctcctgctggccaaactcagcaagggcaagcattggcagctaccgccaacggtattgacagtaataccaaaatcgctcatctgaacggtaaccagtggggctttggctggaacgccggaatcctgtatgaactggataaaaataaccgctatgcactgacctaccgttctgaagtgaaaattgacttcaaaggtaactacagcagcgatcttaatcgtgcgtttaataactacggtttgccaattcctaccgcgacaggtggcgcaacgcaatcgggttatctgacgctgaacctgcctgaaatgtgggaagtgtcaggttataaccgtgttgatccacagtgggcgattcactatagcctggcttacaccagctggagtcagttccagcagctgaaagcgacctcaaccagtggcgacacgctgttccagaaacatgaaggctttaaagatgcttaccgcatcgcgttgggtaccacttattactacgatgataactggaccttccgtaccggtatcgcctttgatgacagcccagttcctgcacagaatcgttctatctccattccggaccaggaccgtttctggctgagtgcaggtacgacttacgcatttaataaagatgcttcagtcgacgttggtgtttcttatatgcacggtcagagcgtgaaaattaacgaaggcccataccagttcgagtctgaaggtaaagcctggctgttcggtactaactttaactacgcgttctga

SEQ ID NO: 29 - DNA, 951 bp, Escherichia coli
ttacaaccagttcttccgcttaaagtacagatacggtgccaggcccgcgaggatcataaagataatcgcgccagggtagccgaagctccacttcagttctggcataaactcaaagttcatgccatagctggaagcaacgagcgtcggcggcaggaataccacggataccaccgagaagattttgatgatgcggttctgctcgatgttgataaaacccattgccgcctgcatcaggaagttcaccttctggaacagggattcgttatgcggcagcagggattcgatatctcgcaggatttcacgcgcctgctccagttgcccacccggtaaacgcgctttacgcaccaggaagttgagcgcgcgctgggtatccatcagacacaggcgaactttccagccgatatcttccagttccgccagagtggagagcgcctcgtcgtactcatcgccctgatgcccttccataatcacccggctcaactgctccaggtcgctatagatattttcaatttcatctgccaactgttcgattttggtttcgaacagatccagcagcaactcgtaggcgttaccgtctaccatcgactggctacgggcacgcatacgatacagacgaaaagcgggcagttcacgctcacgcagagtaaacagacgaccatcacggatggtaaatgccacagtggagttaccggcgtgatcttccgcatcttcaaagaagaagaaggagtgaatatgcaggccgtcgtcgtcttcaaagaaacgtgccgatgcttcgatgtcttccagttccgggcgggttgccaggctctggccaagttcagattgtacgcgcagtcgctcgtcgtcgtccggttcgacaagatcaatccatactgcatttacaaggggttgtgactcttcgacttccagccgggtcagtcggttattttccagttgaaatgcgctcagcat

SEQ ID NO: 30 - DNA, 1407 bp, Escherichia coli
atgcgtacgtttagtggcaaacgtagtacgctggcgctggctatcgccggtgttacagcaatgtcgggctttatggcaatgccggaggctcgcgccgaaggattcatcgacgattcaaccttaaccggcggtatctattactggcagcgtgaacgcgaccgtaaagatgttaccgacggcgacaaatacaaaaccaacctttctcactccacctggaatgccaacctcgattttcagtccggttatgctgctgatatgttcggccttgatattgctgcgtttacggcgattgaaatggcggaaaacggcgacagctcccacccgaacgaaatcgcgttttcaaaaagtaataaagcctatgacgaagactggtccggcgacaaaagcggtataagcctgtataaagctgcggccaaatttaaatacggtccggtttgggcgagggcaggttacattcagccaactggtcaaacgctgttagcgccgcactggagctttatgccaggtacttatcagggggcggaagccggggcgaattttgattacggcgatgctggtgcgttgagtttctcctacatgtggaccaacgaatacaaagcgccgtggcatctggaaatggatgagttttatcagaacgataaaaccaccaaagttgattatctgcactcctttggggcgaaatacgacttcaaaaataacttcgtactggaagcggcatttggtcaggcggaagggtatatcgatcaatattttgccaaagccagctacaaatttgatatcgccggtagcccgttaaccaccagctaccagttctacggtacccgagataaagttgacgatcgcagcgtcaacgacctttatgacggcaccgcctggctgcaagcgttgacctttggttaccgggcggctgacgtagtggatttgcgcctcgaaggcacctgggttaaggctgacggtcagcagggatacttcctgcaacgtatgactccaacctacgcttcctcaaacggtcgcctggatatctggtgggataaccgttctgacttcaacgccaacggcgaaaaagcggtcttcttcggtgcgatgtatgacctgaaaaactggaatcttccaggcttcgccatcggcgcttcctacgtttacgcatgggatgctaaacctgcgacctggcagagcaatccggatgcgtactacgacaaaaaccggactattgaagagtctgcctacagcctggatgcggtctataccattcaggacggtcgcgccaaaggcacgatgttcaaactgcatttcaccgaatacgacaaccactccgacatcccaagctggggcggtggttacggcaacatcttccaggatgagcgtgacgtgaaatttatggtaatcgcaccattcaccatcttctga

SEQ ID NO: 31 - DNA, 1482 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 32 - DNA, 411 bp, Escherichia coli
ttaagagcggttattctgctctttcagcaaatcacgaatttctgtcagtaatacttcttctttagttggtgcaggtgcggctgctggttcttcttttttccgattcagtttgttgattagcttaatcgccataaagatggcaaaggccacaatcagaaaatcaaagacgttttgaatgaagacaccgtaatgcatcacaacagcagggatatccccctgcgcatcgcgtagcgtgacagcaaactgtttaaaatcgatcccgccaattaataagcccagaggaggcatgatgatatcggcaaccagtgaagagacaatcttcccgaatgccgcaccgataatgacacccaccgccaaatccaccacgttcccgcgcatcgcaaattcgcgaaattctttaataatgctcat

SEQ ID NO: 33 - DNA, 1200 bp, Escherichia coli
atgacagagattaactggaaggataatctgcgtattgcctggtttggtaattttctgacaggagccagtatttctttggttgtaccttttatgcccatcttcgtggaaaatctaggtgtagggagtcagcaagtcgctttttatgcaggcttagcaatttctgtctctgctatttccgcggcgctcttttctcctatttggggtattcttgctgacaaatacggccgaaaacccatgatgattcgggcaggtcttgctatgactatcactatgggaggcttggcctttgtcccaaatatctattggttaatctttcttcgtttactaaacggtgtatttgcaggttttgttcctaatgcaacggcactgatagccagtcaggttccaaaggagaaatcaggctctgccttaggtactttgtctacaggcgtagttgcaggtactctaaccggtccctttattggtggctttatcgcagaattatttggcattcgtacagttttcttactggttggtagttttctatttttagctgctattttgactatttgctttatcaaggaagattttcaaccagtagccaaggaaaaggctattccaacaaaggaattatttacctcggttaaatatccctatcttttgctcaatctctttttaaccagttttgtcatccaattttcagctcaatcgatcggccctattttggctctttatgtacgcgacttagggcagacagagaatcttctttttgtctctggtttgattgtgtccagtatgggcttttccagtatgatgagtgcaggagtcatgggcaagctaggtgacaaggtgggcaatcatcgtctcttggttgtcgcccagttttattcagtcatcatctatctcctctgtgccaatgcctctagcccccttcaactaggactctatcgtttcctctttggcttgggaaccggtgccttgattccaggggttaatgccctactcagcaaaatgactcccaaagccggcatttcgagggtctttgccttcaatcaggtattcttttatctgggaggtgttgttggtcccatggcaggttctgcagtagcaggtcaatttggctaccatgctgtcttttatgcgacaagcctttgtgttgcctttagttgtctctttaacctgattcaatttcgaacattattaaaagtaaaggaaatctag

SEQ ID NO: 34 - DNA, 1845 bp, Escherichia coli
tcagaaggtgtagctgccagacaaggtgtattcccgtcctgcagtttggtagccatagactgtctcataatctttgtcgaacaggttggctattttaccacgaactgtcaggtgagaggtgaccggatacgcaaccgcaagatcccacaagctcacaccgcccattttaacggtttgataaggataagatgagtaatccttatcatagcgagtgcctaaatactgataagtaataccccagtcgaagtcatacaactgccagtcgagctggtatttcacctgctgtttagcacggcgtaacaacggcgtgtcggtaatcgcattgcgcgcatcgacataatcataactcacagtatgcgtcagtggtccggtatcaaaattggcggtcgcctcgacgcccttaatccgcgctttcccttcgttgtaatatttcagggtgtgatcatcataatcgatcaagtcactgacatcgttacgatatccggaaatacgccagttcaccccagcggttaagccttcaaacgcgccttcccactgtttgcttttctccgggtccagattcggatttccgtagaagccatacagttgccccagatttggtgccttataagatgtcccgtaggaagcaatgaagcgataaccttcgatgaattcccaaccggcgctggtttgccaggttccatgacgaccaaactgtgagttatcgtcactgcgtgctgcgccttcaaaggtaaaatcgccgacttgttgcagcccggtcagatagatgccggtattacgttgatcatatccatcctcaacataacctgtacccggcgtcgtagtctgtttctgccagtcgacacccgcaccaatactaccgtgaccaacgatgacattgtttgcccactggacggtgtattgcttcatctcatcgagcgtcgccgacgaatcataacgaccataatggggatcgtagttgtaatctttgctatggctatagctggtaatgagttgtgatttaatcagttcgccgttatagcgcagcccggcgtcccaactttggctatagagtttacgggtatcgagcaacggtgaaccgggagaataatacgcgtcataattggtacggttatcatagccatagccgcgcacaaagccgctccaggcatcagtaaagttatgctccagcgcgccataaagcgttttacttaaaaaaccatcgttatctgtctgcgcttgcgttccggtattaccataggcaacaacatcataaccatgagtatgggcataatcgcccaacagcgttacccgtgtcttatcccccagttgttgctgcgtagagacatcatagttctgataactattgcttccccaccctgctgaaatttccgttccgggttcatcgcgcgtcgtgatgatattcaccaccccgcctattgcatcggaaccataaacagcggagcgcggcccacggatatattcaacacgctggacaagcgcaatagggaactggctaaggtcggcagaaccactcacccccgccagattcaggcgtacgccatcaattaacaccaacacatgactggcatttgtaccgcgaataaaaatagatgagagctgacctgaaccgccgttttgggtgatatcgacgcccggaagacggcgcagcacatcattgaccgaggtcgactgccagcggtcgatatcctgacgggtcacaacggtggttggtgcaagcacagtgctgcgcggctgttcaaaacggttagcagtaacgacgagagtatccgggctggtatcctgtgcccaagcggaaaatgccgtgacggaacacgccgtcagcagcgaagcttttttaatcat

SEQ ID NO: 35 - DNA, 1617 bp, Escherichia coli
atgtttagacgaaatcttattacctctgccatcttattaatggcaccgttagccttttctgcacaatcattggctgaatcattaacggtggaacaacgccttgagttattagaaaaggcgttaagagaaacgcaaagcgaactcaaaaagtataaagatgaagagaagaaaaagtatacgccagcgacggtgaatcgtagcgtaagtacgaatgatcaagggtatgccgccaatccgttcccgaccagtagtgccgcaaaacctgatgctgtactggtcaaaaatgaagagaaaaatgccagtgagacaggctcgatttattcttccatgactctgaaagatttcagtaaatttgtgaaagatgaaattggctttagttacaacggctattaccgttctggttgggggaccgcctctcatggttcacctaaatcatgggcgattggttctctgggccgctttggtaacgaatactccggctggtttgatttgcagttaaaacaacgtgtctacaacgaaaacggcaaacgggttgatgccgttgtgatgatggatggtaacgttggtcagcagtactctaccggctggtttggcgataacgccggtggcgagaactatatgcagttctccgatatgtacgttaccaccaaaggtttcctgccctttgcgccagaggctgatttctgggtgggtaaacacggtgcgccgaaaattgaaatccagatgcttgactggaaaacgcagcgtactgatgccgcagcgggtgtaggtctggaaaactggaaagtcggtccgggtaaaattgatatcgcgctggttcgcgaagatgtcgatgattacgatcgcagcctgcaaaacaaacagcagattaataccaataccattgatttacgctataaagatatcccgttatgggataaagccaccttaatggtaagtggtcgttatgtcacggcaaacgaaagcgcatcggaaaaagataatcaggataataacgggtattatgactggaaagatacctggatgtttggcacatctttaacgcagaaatttgataaaggtggcttcaacgaattctccttcctggtcgcgaataactctatcgccagtaactttggccgttatgctggcgcaagtccatttaccacctttaatggtcgttattatggtgatcacaccggcggaacagcggtacgtctgacttcgcagggcgaagcctatattggcgatcatttcattgtagctaacgcgattgtttactccttcggtaacgatatatatagctacgaaacaggcgcccactctgatttcgaatctattcgtgcggttgttcgcccggcctatatttgggaccaatataaccagacaggtgttgaactgggctatttcacccagcaaaacaaagatgcgaatagtaataaatttaatgagtctggttataaaaccacgctcttccatacctttaaagtcaataccagtatgttgacctcgcgtccggaaattcgtttctacgccacgtatatcaaagccctggaaaacgaactggatggcttcacctttgaagacaataaagacgaccagtttgctgtcggtgcccaggctgaaatctggtggtaa

SEQ ID NO: 36 - protein, 747 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 37 - protein, 727 aa, Escherichia coli
MARSKTAQPKHSLRKIAVVVATAVSGMSVYAQAAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 38 - protein, 707 aa, Escherichia coli
MARSKTAQPKHSLRKIAVVVATAVSGMSVYAQATPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 39 - protein, 683 aa, Escherichia coli
MARSKTAQPKHSLRKIAVVVATAVSGMSVYAQASVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 40 - protein, 642 aa, Escherichia coli
MARSKTAQPKHSLRKIAVVVATAVSGMSVYAQALNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 41 - protein, 587 aa, Escherichia coli
MARSKTAQPKHSLRKIAVVVATAVSGMSVYAQALKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 42 - protein, 281 aa, Escherichia coli
MSQTSTLKGQCIAEFLGTGLLIFFGVGCVAALKVAGASFGQWEISVIWGLGVAMAIYLTAGVSGAHLNPAVTIALWLFACFDKRKVIPFIVSQVAGAFCAAALVYGLYYNLFFDFEQTHHIVRGSVESVDLAGTFSTYPNPHINFVQAFAVEMVITAILMGLILALTDDGNGVPRGPLAPLLIGLLIAVIGASMGPLTGFAMNPARDFGPKVFAWLAGWGNVAFTGGRDIPYFLVPLFGPIVGAIVGAFAYRKLIGRHLPCDICVVEEKETTTPSEQKASL

SEQ ID NO: 43 - protein, 362 aa, Escherichia coli
MMKRNILAVIVPALLVAGTANAAEIYNKDGNKVDLYGKAVGLHYFSKGNGENSYGGNGDMTYARLGFKGETQINSDLTGYGQWEYNFQGNNSEGADAQTGNKTRLAFAGLKYADVGSFDYGRNYGVVYDALGYTDMLPEFGGDTAYSDDFFVGRVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTYVDYIINQIDSDNKLGVGSDDTVAVGIVYQF

SEQ ID NO: 44 - protein, 346 aa, Escherichia coli
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 45 - protein, 301 aa, Escherichia coli
MKKLLPCTALVMCAGMACAQAEERNDWHFNIGAMYEIENVEGYGEDMDGLAEPSVYFNAANGPWRIALAYYQEGPVDYSAGKRGTWFDRPELEVHYQFLENDDFSFGLTGGFRNYGYHYVDEPGKDTANMQRWKIAPDWDVKLTDDLRFNGWLSMYKFANDLNTTGYADTRVETETGLQYTFNETVALRVNYYLERGFNMDDSRNNGEFSTQEIRAYLPLTLGNHSVTPYTRIGLDRWSNWDWQDDIEREGHDFNRVGLFYGYDFQNGLSVSLEYAFEWQDHDEGDSDKFHYAGVGVNYSF

SEQ ID NO: 46 - protein, 367 aa, Escherichia coli
MKVKVLSLLVPALLVAGAANAAEVYNKDGNKLDLYGKVDGLHYFSDNKDVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGNPSGEGFTSGVTNNGRDALRQNGDGVGGSITYDYEGFGIGGAISSSKRTDAQNTAAYIGNGDRAETYTGGLKYDANNIYLAAQYTQTYNATRVGSLGWANKAQNFEAVAQYQFDFGLRPSLAYLQSKGKNLGRGYDDEDILKYVDVGATYYFNKNMSTYVDYKINLLDDNQFTRDAGINTDNIVALGLVYQF

SEQ ID NO: 47 - protein, 230 aa, Escherichia coli
MKKINAIILLSSLTSASVFAGAYVENREAYNLASDQGEVMLRVGYNFDMGAGIMLTNTYNFQREDELKHGYNEIEGWYPLFKPTDKLTIQPGGLINDKSIGSGGAVYLDVNYKFVPWFNLTVRNRYNHNNYSSTDLSGELDNNDTYEIGTYWNFKITDKFSYTFEPHYFMRVNDFNSSNGKDHHWEITNTFRYRINEHWLPYFELRWLDRNVEPYHREQNQIRIGTKYFF

SEQ ID NO: 48 - protein, 317 aa, Escherichia coli
MRAKLLGIVLTTPIAISSFASTETLSFTPDNINADISLGTLSGKTKERVYLAEEGGRKVSQLDWKFNNAAIIKGAINWDLMPQISIGAAGWTTLGSRGGNMVDQDWMDSSNPGTWTDESRHPDTQLNYANEFDLNIKGWLLNEPNYRLGLMAGYQESRYSFTARGGSYIYSSEEGFRDDIGSFPNGERAIGYKQRFKMPYIGLTGSYRYEDFELGGTFKYSGWVESSDNDEHYDPGKRITYRSKVKDQNYYSVAVNAGYYVTPNAKVYVEGAWNRVTNKKGNTSLYDHNNNTSDYSKNGAGIENYNFITTAGLKYTF

SEQ ID NO: 49 - protein, 351 aa, Escherichia coli
MKKSTLALVVMGIVASASVQAAEIYNKDGNKLDVYGKVKAMHYMSDNASKDGDQSYIRFGFKGETQINDQLTGYGRWEAEFAGNKAESDTAQQKTRLAFAGLKYKDLGSFDYGRNLGALYDVEAWTDMFPEFGGDSSAQTDNFMTKRASGLATYRNTDFFGVIDGLNLTLQYQGKNENRDVKKQNGDGFGTSLTYDFGGSDFAISGAYTNSDRTNEQNLQSRGTGKRAEAWATGLKYDANNIYLATFYSETRKMTPITGGFANKTQNFEAVAQYQFDFGLRPSLGYVLSKGKDIEGIGDEDLVNYIDVGATYYFNKNMSAFVDYKINQLDSDNKLNINNDDIVAVGMTYQF

SEQ ID NO: 50 - protein, 446 aa, Escherichia coli [sequence omitted]

SEQ ID NO: 51 - protein, 774 aa, Escherichia coli
MTPLRVFRKTTPLVNTIRLSLLPLAGLSFSAFAAQVNIAPGSLDKALNQYAAHSGFTLSVDASLTRGKQSNGLHGDYDVESGLQQLLDGSGLQVKPLGNNSWTLEPAPAPKEDALTVVGDWLGDARENDVFEHAGARDVIRREDFAKTGATTMREVLNRIPGVSAPENNGTGSHDLAMNFGIRGLNPRLASRSTVLMDGIPVPFAPYGQPQLSLAPVSLGNMDAIDVVRGGGAVRYGPQSVGGVVNFVTRAIPQDFGIEAGVEGQLSPTSSQNNPKETHNLMVGGTADNGFGTALLYSGTRGSDWREHSATRIDDLMLKSKYAPDEVHTFNSLLQYYDGEADMPGGLSRADYDADRWQSTRPYDRFWGRRKLASLGYQFQPDSQHKFNIQGFYTQTLRSGYLEQGKRITLSPRNYWVRGIEPRYSQIFMIGPSAHEVGVGYRYLNESTHEMRYYTATSSGQLPSGSSPYDRDTRSGTEAHAWYLDDKIDIGNWTITPGMRFEHIESYQNNAITGTHEEVSYNAPLPALNVLYHLTDSWNLYANTEGSFGTVQYSQIGKAVQSGNVEPEKARTWELGTRYDDGALTAEMGLFLINFNNQYDSNQTNDTVTARGKTRHTGLETQARYDLGTLTPTLDNVSIYASYAYVNAEIREKGDTYGNLVPFSPKHKGTLGVDYKPGNWTFNLNSDFQSSQFADNANTVKESADGSTGRIPGFMLWGARVAYDFGPQMADLNLAFGVKNIFDQDYFIRSYDDNNKGIYAGQPRTLYMQGSLKF

SEQ ID NO: 52 - protein, 417 aa, Escherichia coli [sequence omitted]

SEQ ID NO: 53 - protein, 1049 aa, Escherichia coli [sequence omitted]

SEQ ID NO: 54 - protein, 262 aa, Halobacterium sp.
MLELLPTAVEGVSQAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATSD

SEQ ID NO: 55 - protein, 496 aa, Aeromonas salmonicida
MTMFKSKLALAVALGLGMSSPLWAAEGGIEARLAALEARVQAAEARAEAAESRASQAEIKASAASEEAQVANTRSQKVDEKTAGAQGFEFHGYARSGLLVNGNGNGGRGGPYITPAGSVGGAVGRLGNEDDTYMEANLLKTQTFDDGSWARYKLMLADGVETSNDWTASDSSLNTRQVFAEIGNLASFDGAFKNAVLWAGKRFDRDNFDIHWLDSDVVFLAGTGGGVYDVQLADSWKVNFSLYGRDYGDISASGLSDIESYILTMNNRFGNWQWMVNGLLANDNEQRINDGGKGSEAADKGAHTLVAYHADSFFGINPGFSKAAILYGHGLGAELKGLGSDSELLPDADAVRVAVFGATDLNPRWRFAPALLAEQSQDRYVKGDEYQWLTLNTRVAQVLSQNVELVYEAAWQYMDLDPKGYKGRQAVNGNFTKLTFAPTFKAQTAGFFERPELRVFASWMDWSSELDSYASDDAMGQSNFTAGGEWNFGVQMETWF

SEQ ID NO: 56 - protein, 746 aa, Escherichia coli
MNKKIHSLALLVNLGIYGVAQAQEPTDTPVSHDDTIVVTAAEQNLQAPGVSTITADEIRKNPVARDVSKIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDSYTRSKYGDETNRLYRQNYALTWNGGWDNGVTTSNWVQYEHTRNSRIPEGLAGGTEGKFNEKATQDFVDIDLDDVMLHSEVNLPIDFLVNQTLTLGTEWNQQRMKDLSSNTQALTGTNTGGAIDGVSTTDRSPYSKAEIFSLFAENNMELTDSTIVTPGLRFDHHSIVGNNWSPALNISQGLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSVNTHF

SEQ ID NO: 57 - protein, 729 aa, Escherichia coli
MLSTQFNRDNQYQAITKPSLLAGCIALALLPSAAFAAPATEETVIVEGSATAPDDGENDYSVTSTSAGTKMQMTQRDIPQSVTIVSQQRMEDQQLQTLGEVMENTLGISKSQADSDRALYYSRGFQIDNYMVDGIPTYFESRWNLGDALSDMALFERVEVVRGATGLMTGTGNPSAAINMVRKHATSREFKGDVSAEYGSWNKERYVADLQSPLTEDGKIRARIVGGYQNNDSWLDRYNSEKTFFSGIVDADLGDLTTLSAGYEYQRIDVNSPTWGGLPRWNTDGSSNSYDRARSTAPDWAYNDKEINKVFMTLKQQFADTWQATLNATHSEVEFDSKMMYVDAYVNKADGMLVGPYSNYGPGFDYVGGTGWNSGKRKVDALDLFADGSYELFGRQHNLMFGGSYSKQNNRYFSSWANIFPDEIGSFYNFNGNFPQTDWSPQSLAQDDTTHMKSLYAATRVTLADPLHLILGARYTNWRVDTLTYSMEKNHTTPYAGLVFDINDNWSTYASYTSIFQPQNDRDSSGKYLAPITGNNYELGLKSDWMNSRLTTTLAIFRIEQDNVAQSTGTPIPGSNGETAYKAVDGTVSKGVEFELNGAITDNWQLTFGATRYIAEDNEGNAVNPNLPRTTVKMFTSYRLPVMPELTVGGGVNWQNRVYTDTVTPYGTFRAEQGSYALVDLFTRYQVTKNFSLQGNVNNLFDKTYDTNVEGSIVYGTPRNFSITGTYQF

SEQ ID NO: 58 - protein, 452 aa, Escherichia coli
MQTVLAKIVADKAIWVEARKQQQPLASFQNEVQPSTRHFYDALQGARTAFILECKKASPSKGVIRDDFDPARIAAIYKHYASAISVLTDEKYFQGSFNFLPIVSQIAPQPILCKDFIIDPYQIYLARYYQADACLLMLSVLDDDQYRQLAAVAHSLEMGVLTEVSNEEEQERAIALGAKVVGINNRDLRDLSIDLNRTRELAPKLGHNVTVISESGINTYAQVRELSHFANGFLIGSALMAHDDLHAAVRRVLLGENKVCGLTRGQDAKAAYDAGAIYGGLIFVATSPRCVNVEQAQEVMAAAPLQYVGVFRNHDIADVVDKAKVLSLAAVQLHGNEEQLYIDTLREALPAHVAIWKALSVGETLPAREFQHVDKYVLDNGQGGSGQRFDWSLLNGQSLGNVLLAGGLGADNCVEAAQTGCAGLDFNSAVESQPGIKDARLLASVFQTLRAY

SEQ ID NO: 59 - protein, 289 aa, Escherichia coli
MRTLQGWLLPVFMLPMAVYAQEATVKEVHDAPAVRGSIIANMLQEHDNPFTLYPYDTNYLIYTQTSDLNKEAIASYDWAENARKDEVKFQLSLAFPLWRGILGPNSVLGASYTQKSWWQLSNSEESSPFRETNYEPQLFLGFATDYRFAGWTLRDVEMGYNHDSNGRSDPTSRSWNRLYTRLMAENGNWLVEVKPWYVVGNTDDNPDITKYMGYYQLKIGYHLGDAVLSAKGQYNWNTGYGGAELGLSYPITKHVRLYTQVYSGYGESLIDYNFNQTRVGVGVMLNDLF

SEQ ID NO: 60 - protein, 663 aa, Escherichia coli
MFRLNPFVRVGLCLSAISCAWPVLAVDDDGETMVVTASSVEQNLKDAPASISVITQEDLQRKPVQNLKDVLKEVPGVQLTNEGDNRKGVSIRGLDSSYTLILVDGKRVNSRNAVFRHNDFDLNWIPVDSIERIEVVRGPMSSLYGSDALGGVVNIITKKIGQKWSGTVTVDTTIQEHRDRGDTYNGQFFTSGPLIDGVLGMKAYGSLAKREKDDPQNSTTTDTGETPRIEGFSSRDGNVEFAWTPNQNHDFTAGYGFDRQDRDSDSLDKNRLERQNYSVSHNGRWDYGTSELKYYGEKVENKNPGNSSPITSESNTVDGKYTLPLTAINQFLTVGGEWRHDKLSDAVNLTGGTSSKTSASQYALFVEDEWRIFEPLALTTGVRMDDHETYGEHWSPRAYLVYNATDTVTVKGGWATAFKAPSLLQLSPDWTSNSCRGACKIVGSPDLKPETSESWELGLYYMGEEGWLEGVESSVTVFRNDVKDRISISRTSDVNAAPGYQNFVGFETGANGRRIPVFSYYNVNKARIQGVETELKIPFNDEWKLSINYTYNDGRDVSNGENKPLSDLPFHTANGTLDWKPLALEDWSFYVSGHYTGQKRADSATAKTPGGYTIWNTGAAWQVTKDVKLRAGVLNLGDKDLSRDDYSYNEDGRRYFMAVDYRF

SEQ ID NO: 61 - protein, 1120 aa, Escherichia coli
MTMFQYYKRSRHFVFSAFIAFVFVLLCQNTAFARASSNGDLPTKADLQAQLDSLNKQKDLSAQDKLVQQDLTDTLATLDKIDRIKEETVQLRQKVAEAPEKMRQATAALTALSDVDNDEETRKILSTLSLRQLETRVAQALDDLQNAQNDLASYNSQLVSLQTQPERVQNAMYNASQQLQQIRSRLDGTDVGETALRPSQKVLMQAQQALLNAEIDQQRKSLEGNTVLQDTLQKQRDYVTANSARLEHQLQLLQEAVNSKRLTLTEKTAQEAVSPDEAARIQANPLVKQELEINQQLSQRLITATENGNQLMQQNIKVKNWLERALQSERNIKEQIAVLKGSLLLSRILYQQQQTLPSADELENMTNRIADLRLEQFEVNQQRDALFQSDAFVNKLEEGHTNEVNSEVHDALLQVVDMRRELLDQLNKQLGNQLMMAINLQINQQQLMSVSKNLKSILTQQIFWVNSNRPMDWDWIKAFPQSLKDEFKSMKITVNWQKAWPAVFIAFLAGLPLLLIAGLIHWRLGWLKAYQQKLASAVGSLRNDSQLNTPKAILIDLIRALPVCLIILAVGLILLTMQLNISELLWSFSKKLAIFWLVFGLCWKVLEKNGVAVRHFGMPEQQTSHWRRQIVRISLALLPIHFWSVVAELSPLHLMDDVLGQAMIFFNLLLIAFLVWPMCRESWRDKESHTMRLVTITVLSIIPIALMVLTATGYFYTTLRLAGRWIETVYLVIIWNLLYQTVLRGLSVAARRIAWRRALARRQNLVKEGAEGAEPPEEPTIALEQVNQQTLRITMLLMFALFGVMFWAIWSDLITVFSYLDSITLWHYNGTEAGAAVVKNVTMGSLLFAIIASMVAWALIRNLPGLLEVLVLSRLNMRQGASYAITTILNYIIIAVGAMTVFGSLGVSWDKLQWLAAALSVGLGFGLQEIFGNFVSGLIILFERPVRIGDTVTIGSFSGTVSKIRIRATTITDFDRKEVIIPNKAFVTERLINWSLTDTTTRLVIRLGVAYGSDLEKVRKVLLKAATEHPRVMHEPMPEVFFTAFGASTLDHELRLYVRELRDRSRTVDELNRTIDQLCRENDINIAFNQLEVHLHNEKGDEVTEVKRDYKGDDPTPAVG

SEQ ID NO: 62 - protein, 448 aa, Escherichia coli
MVMSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 63 - protein, 316 aa, Escherichia coli
MLSAFQLENNRLTRLEVEESQPLVNAVWIDLVEPDDDERLRVQSELGQSLATRPELEDIEASARFFEDDDGLHIHSFFFFEDAEDHAGNSTVAFTIRDGRLFTLRERELPAFRLYRMRARSQSMVDGNAYELLLDLFETKIEQLADEIENIYSDLEQLSRVIMEGHQGDEYDEALSTLAELEDIGWKVRLCLMDTQRALNFLVRKARLPGGQLEQAREILRDIESLLPHNESLFQKVNFLMQAAMGFINIEQNRIIKIFSVVSVVFLPPTLVASSYGMNFEFMPELKWSFGYPGAIIFMILAGLAPYLYFKRKNWL

SEQ ID NO: 64 - protein, 468 aa, Escherichia coli
MRTFSGKRSTLALAIAGVTAMSGFMAMPEARAEGFIDDSTLTGGIYYWQRERDRKDVTDGDKYKTNLSHSTWNANLDFQSGYAADMFGLDIAAFTAIEMAENGDSSHPNEIAFSKSNKAYDEDWSGDKSGISLYKAAAKFKYGPVWARAGYIQPTGQTLLAPHWSFMPGTYQGAEAGANFDYGDAGALSFSYMWTNEYKAPWHLEMDEFYQNDKTTKVDYLHSFGAKYDFKNNFVLEAAFGQAEGYIDQYFAKASYKFDIAGSPLTTSYQFYGTRDKVDDRSVNDLYDGTAWLQALTFGYRAADVVDLRLEGTWVKADGQQGYFLQRMTPTYASSNGRLDIWWDNRSDFNANGEKAVFFGAMYDLKNWNLPGFAIGASYVYAWDAKPATWQSNPDAYYDKNRTIEESAYSLDAVYTIQDGRAKGTMFKLHFTEYDNHSDIPSWGGGYGNIFQDERDVKFMVIAPFTIF

SEQ ID NO: 65 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 66 - protein, 86 aa, Escherichia coli
MTTTGLRPRLNVRQRKDTGYLPHSSPFSLQFRPAILYSDGYLPLVPEDKNETDKIHTPRIVPQKLERTPSDTSRSRGCHCFYAGWL

SEQ ID NO: 67 - protein, 72 aa, Escherichia coli [sequence omitted]

SEQ ID NO: 68 - protein, 136 aa, Escherichia coli
MSIIKEFREFAMRGNVVDLAVGVIIGAAFGKIVSSLVADIIMPPLGLLIGGIDFKQFAVTLRDAQGDIPAVVMHYGVFIQNVFDFLIVAFAIFMAIKLINKLNRKKEEPAAAPAPTKEEVLLTEIRDLLKEQNNRS

SEQ ID NO: 69 - protein, 201 aa, Escherichia coli
MGKIIGITGGIASGKSTVTNFLRQQGFQAVDADAVVHQLQKPGGRLFEALVQHFGQEIILENGELNRPLLASLIFSNPEEQKWSNQIQGEIIREELATLREQLAQTEEIFFMDIPLLFEQDYSDWFAETWLVYVDRDAQVERLMKRDQLSKDEAESRLAAQWPLEKKKDLASQVLDNNGNQNQLLNQVHILLEGGRQDDRD

SEQ ID NO: 70 - protein, 399 aa, Escherichia coli
MTEINWKDNLRIAWFGNFLTGASISLVVPFMPIFVENLGVGSQQVAFYAGLAISVSAISAALFSPIWGILADKYGRKPMMIRAGLAMTITMGGLAFVPNIYWLIFLRLLNGVFAGFVPNATALIASQVPKEKSGSALGTLSTGVVAGTLTGPFIGGFIAELFGIRTVFLLVGSFLFLAAILTICFIKEDFQPVAKEKAIPTKELFTSVKYPYLLLNLFLTSFVIQFSAQSIGPILALYVRDLGQTENLLFVSGLIVSSMGFSSMMSAGVMGKLGDKVGNHRLLVVAQFYSVIIYLLCANASSPLQLGLYRFLFGLGTGALIPGVNALLSKMTPKAGISRVFAFNQVFFYLGGVVGPMAGSAVAGQFGYHAVFYATSLCVAFSCLFNLIQFRTLLKVKEI

SEQ ID NO: 71 - protein, 285 aa, Escherichia coli
MATKLQDGNTPCLAATPSEPRPTVLVFDSGVGGLSVYDEIRHLLPDLHYIYAFDNVAFPYGEKSEAFIVERVVAIVTAVQERYPLALAVVACNTASTVSLPALREKFDFPVVGVVPAIKPAARLTANGIVGLLATRGTVKRSYTHELIARFANECQIEMLGSAEMVELAEAKLHGEDVSLDALKRILRPWLRMKEPPDTVVLGCTHFPLLQEELLQVLPEGTRLVDSGAAIARRTAWLLEHEAPDAKSADANIAFCMAMTPGAEQLLPVLQRYGFETLEKLAVLG

SEQ ID NO: 72 - protein, 614 aa, Escherichia coli
MIKKASLLTACSVTAFSAWAQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDEPGTEISAGWGSNSYQNYDVSTQQQLGDKTRVTLLGDYAHTHGYDVVAYGNTGTQAQTDNDGFLSKTLYGALEHNFTDAWSGFVRGYGYDNRTNYDAYYSPGSPLLDTRKLYSQSWDAGLRYNGELIKSQLITSYSHSKDYNYDPHYGRYDSSATLDEMKQYTVQWANNVIVGHGSIGAGVDWQKQTTTPGTGYVEDGYDQRNTGIYLTGLQQVGDFTFEGAARSDDNSQFGRHGTWQTSAGWEFIEGYRFIASYGTSYKAPNLGQLYGFYGNPNLDPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLSGSYTF

SEQ ID NO: 73 - protein, 538 aa, Escherichia coli
MFRRNLITSAILLMAPLAFSAQSLAESLTVEQRLELLEKALRETQSELKKYKDEEKKKYTPATVNRSVSTNDQGYAANPFPTSSAAKPDAVLVKNEEKNASETGSIYSSMTLKDFSKFVKDEIGFSYNGYYRSGWGTASHGSPKSWAIGSLGRFGNEYSGWFDLQLKQRVYNENGKRVDAVVMMDGNVGQQYSTGWFGDNAGGENYMQFSDMYVTTKGFLPFAPEADFWVGKHGAPKIEIQMLDWKTQRTDAAAGVGLENWKVGPGKIDIALVREDVDDYDRSLQNKQQINTNTIDLRYKDIPLWDKATLMVSGRYVTANESASEKDNQDNNGYYDWKDTWMFGTSLTQKFDKGGFNEFSFLVANNSIASNFGRYAGASPFTTFNGRYYGDHTGGTAVRLTSQGEAYIGDHFIVANAIVYSFGNDIYSYETGAHSDFESIRAVVRPAYIWDQYNQTGVELGYFTQQNKDANSNKFNESGYKTTLFHTFKVNTSMLTSRPEIRFYATYIKALENELDGFTFEDNKDDQFAVGAQAEIWW

SEQ ID NO: 74 - protein, 398 aa, Escherichia coli
MELNERNITMNIKIAALTLAIASGISAQWAIAADMPASPAPTIPVKQYVTQVNADNSVTFRYFAPGAKNVSVVVGVPVPDNIHPMTKDEAGVWSWRTPILKGNLYEYFFNVDGVRSIDTGTAMTNPQRQVNSSMILVPGSYLDTRSVAHGDLIAITYHSNALQSERQMYVWTPPGYTGMGEPLPVLYFYHGFGDTGRSAIDQGRIPQIMDNLLAEGKIKPMLVVIPDTETDAKGIIPEDFVPQERRKVFYPLNAKAADRELMNDIIPLISKRFNVRKDADGRALAGLSQGGYQALVSGMNHLESFGWLATFSGVTTTTVPDEGVAARLNDPAAINQQLRNFTVVVGDKDVVTGKDIAGLKTELEQKKINFDYQEYPGLNHEMDVWRPAYAAFVQKLFK

SEQ ID NO: 75 - DNA, 33 bp, Artificial sequence
  Primer
accggaattccggatggcgcgttccaaaactgc

SEQ ID NO: 76 - DNA, 40 bp, Artificial sequence
  Primer
catgccatggctcgagttagaaacggaaggttgcggttgc

SEQ ID NO: 77 - DNA, 30 bp, Artificial sequence
  Primer
agcaggcccccatgctgcctgtgcataaac

SEQ ID NO: 78 - DNA, 29 bp, Artificial sequence
  Primer
ctttttgaatcggcgttgcctgtgcataa

SEQ ID NO: 79 - DNA, 30 bp, Artificial sequence
  Primer
agcgcttcttttaccgatgcctgtgcataa

SEQ ID NO: 80 - DNA, 30 bp, Artificial sequence
  Primer
cttcaggccattcagtgcctgtgcataaac

SEQ ID NO: 81 - DNA, 30 bp, Artificial sequence
  Primer
ctgaacttctttcagtgcctgtgcataaac

SEQ ID NO: 82 - DNA, 30 bp, Artificial sequence
  Primer
gtttatgcacaggcagcatgggggcctgct

SEQ ID NO: 83 - DNA, 31 bp, Artificial sequence
  Primer
gtttatgcacaggcaacgccgattcaaaaag

SEQ ID NO: 84 - DNA, 30 bp, Artificial sequence
  Primer
ttatgcacaggcatcggtaaaagaagcgct

SEQ ID NO: 85 - DNA, 30 bp, Artificial sequence
  Primer
gtttatgcacaggcactgaatggcctgaag

SEQ ID NO: 86 - DNA, 30 bp, Artificial sequence
  Primer
gtttatgcacaggcactgaaagaagttcag

(19) United States
(12) Patent Application Publication
(10) Pub. No.: US 2010/0189777 A1
(43) Pub. Date: Jul. 29, 2010

(54) FUNCTIONALIZED NANOCOMPARTMENTS WITH A TRANSPORT SYSTEM

(75) Inventors: Ulrich Schwaneberg, Bremen (DE); Ozana Onaca, Basel (CH); Prasenjit Sarkar, Raleigh, NC (US); Bernhard Hauer, Fussgonheim (DE); Thomas Friedrich, Darmstadt (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/669,581

(22) PCT Filed: Jul. 7, 2008
(86) PCT No.: PCT/EP08/58735

§ 371 Date: Jan. 19, 2010

(30) Foreign Application Priority Data
Jul. 20, 2007 (EP) 07112838.3

(51) Int. Cl. A61K 9/127; B32B 15/02; B01J 13/02

(52) U.S. Cl. 424/450; 428/402.2; 427/213.3

(57) ABSTRACT

The invention is directed to vesicles comprising at least one transmembrane triggered transport system for controlled transmembrane efflux and/or influx.

The invention is directed to vesicles comprising at least one transmembrane triggered transport system for controlled transmembrane efflux and/or influx.

Vesicles are spherically shaped cages composed of self-assembled amphiphilic molecules. Amphiphiles are macromolecules that consist of two components that differ in their affinity for solutes. The hydrophobic part of the amphiphile prefers non-polar solvents whereas the hydrophilic one has affinity for aqueous medium. Alternative vesicles can be formed by macromolecules with three connected parts hydrophilic-hydrophobic-hydrophilic. These structural features permit the aggregation of hydrophobic segments in a selective solvent. Therefore these molecules self-assemble and form ordered structures (micelles, rods, vesicles or larger aggregates) in aqueous environment.

Vesicles are mainly used for encapsulation, e.g. as drug carriers, or for building compartments for controlled reactions.

Many approaches are known for the synthesis of vesicle in order to obtain the desired functionality in view of membrane stability, flexibility or permeability for example.

Liposomes are vesicles made of phospholipids which are amphiphilic molecules. Polymer vesicles, often referred as “polymersomes”, have been studied in detail and progress has been summarized in reviews [Discher et al., Science 2002, 297; 967 973]. Polymersomes or polymer vesicles consist of self-assembled di- or triblock copolymers.
The Synthosome, which is a functionalized nanocompartment system, has been developed for putative biotechnological applications [Nardin et al., Chem. Commun. 2000, 1433 1434]. A Synthosome is a hollow sphere consisting of a mechanically stable vesicle with a block copolymer membrane and an engineered transmembrane protein acting as the selective gate.

The Synthosomes are formed out of block copolymers by self-assembly in presence of a trans-membrane protein. Synthosome system combine in a biomimetic approach the unmatched selectivity of biological transport systems with the robustness of polymers to novel functionalised sieves. Synthosomes are mechanically more stable than liposomes and more selective than conventional functionalised beads or polymerosomes.

Transmembrane proteins such as OmpF, Maltoporin, and FhuA were incorporated into the polymeric membrane and used as gates for selective compound passage.

As disclosed in EP 1559790A1 these Synthosomes are used as novel separation principle to separate targeted compound by entrapment or to subject them to a specific reaction, for example enzymatic conversion [Onaca et al., Biotechnol. J., 2006, 795-805, Nallani et al., J. Biotech., 2006, 50-59].

The selection is based on the concordance in size of the compound to be passage and the diameter of the aperture of the pore formed by the embedded transmembrane protein or interaction of the transmembrane pore with the translocating compound.

There is still a need for a control system, e.g. a trigger for the transmembrane passage of compounds.

It is object of the present invention to put at disposal such a control system, e.g. a trigger for the transmembrane passage of compounds, which allows at a predetermined time translocation through the pore.

The passage should be possible only for defined compounds, in other word a preference (selection or separation) should take place.
It is further object of the present invention that the trigger does not only control the moment of the transmembrane transport but additionally the velocity of the translocation, e.g. the amount of compound efflux/influx in a certain time.

It was now found that this object is achieved by providing the transmembrane transport trigger system and the process according to the invention described herein and the embodiments characterized in the claims.

In one embodiment the subject matter of the present invention is a vesicles comprising at least one transmembrane transport trigger system.

According to the present invention the term “vesicles” defines semi-permeable spherically shaped cages composed of self-assembled amphiphilic molecules in water or aqueous solution. In other words a vesicle represents an essentially a non- or semi-permeable bag of aqueous solution as surrounded by a self-assembled, stable membrane composed predominantly, by mass, of self-assembled amphiphilic molecules.

It is essential for the present invention that the semi-permeability is a selective permeability to solutes, preferably water is achieved through the transmembran triggered system (biological component).
The vesicle membrane of the invention prevents compound fluxes through the membrane and accommodates the transmembrane triggered transport system in functional form.

In one embodiment of the present invention the vesicle is selected from the group consisting of: liposomes, polymersomes and Synthosomes.

In fact the invention relates to all kinds of vesicles, even those comprising biomembranes, as far as they show the selective permeability to solutes, preferably water.
In one embodiment, the invention provides vesicles that achieve the selective permeability to solutes of amphiphilic molecules in the membrane and preferably by covalently cross-linked molecules.

In one embodiment the vesicle according to the invention consists of a encapsulating membrane.

Encapsulating membranes as used in the present invention compartmentalize by being selectively permeable to solutes, either contained inside or maintained outside of the spatial volume delimited by the membrane. Thus, the vesicle of the invention is a capsule in a, preferably aqueous, solution, which also contains the, preferably aqueous, solution.

In one embodiment the vesicle according to the invention is a Synthosome whereby the encapsulating membrane is composed of block copolymers.

Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent.
Vesicles composed of block copolymers are disclosed by Nardin et al., [Eur. Physical one. J. E 4, (2001), 403-410] as well as in WO 01/32146.

In one embodiment the amphiphilic copolymer is a segmented copolymer with at least a hydrophilic region A and at least a hydrophobic region B, whereby the segmented copolymers is self-assembling under formation of vesicles. The amphiphilic copolymer can contain also more than one hydrophilic and more than one hydrophobic section or region. Preferably, the copolymer can has a ABA structure with two hydrophilic and one hydrophobic section localized between them. For the synthesis of vesicles according to invention block copolymers are preferably used, preferably linear block copolymers as well as in addition grafted copolymers and/or comb structures copolymer, which possess at least one hydrophilic and at least one hydrophobic section.

In one embodiment the amphiphilic copolymer is select from the group consisting of:

ABA-block copolymer: poly(2-methyloxazoline)-poly(dimethylsiloxane)-poly(2-methyloxazoline) (PMOXA-PDMS-PMOXA); poly(ethylene glycol)-poly-(propylene sulfide)-poly(ethylene glycol) (PEG-PPS-PEG); AB-block copolymers: poly(ethyleneoxide)-poly(ethylethylene) (PEO-PEE), poly(styrene)-poly(3-(isocyano-L-alanyl-amino-ethyl)-thiophene) (PS-PIAI); and polyelectrolyte system: poly(styrenesulfate), poly(allylamine) (PSS, PAH).

In one embodiment the amphiphilic copolymer is the ABA-block copolymer: poly(2-methyloxazoline)-poly(dimethylsiloxane)-poly(2-methyloxazoline) (PMOXA-PDMS-PMOXA).

In one embodiment the amphiphilic copolymer is selected from the group as disclosed in EP 1559790 A1, paragraphs [0011] to [0015].

In one embodiment the thickness of the vesicles walls ranges between 5 nm and 100 nm, preferably between 5 nm and 50 nm, between 6 nm and 30 nm, between 7 nm and 25 nm, between 8 nm and 15 nm, more preferably 10 nm. In one embodiment the diameter of the vesicles walls ranges between 10 and 2500 nm, 20 nm and 1000 nm, between 40 nm and 800 nm, between 50 nm and 400 nm, between 50 nm and 200 nm, 5 nm and 150 nm, between 200 nm and 400 nm, between 100 nm and 400 nm, more preferably 100 nm and 200 nm.

In one embodiment the vesicles of the invention are characterized by size exclusion chromatography, zeta potential measurement, microscopy and/or differential scanning calorimetric.

In one embodiment the vesicles of the invention comprises a transmembrane efflux and/or influx trigger system.

In other words, the trigger system allows the controlled transmembrane efflux from the vesicle and/or the controlled transmembrane influx into the vesicle at a predetermined time.
Furthermore, the trigger system allows the controlled transmembrane efflux and/or influx of certain defined and selected compounds.

In one embodiment the vesicles of the invention comprises at least one transmembrane channel protein, preferably a pore forming protein.

The transmembrane channel protein is selected from the group consisting of polypeptides with the activity of:
a) a pore-forming transmembrane protein,
b) a pore-forming transmembrane protein with a alpha-helical transmembrane structure, in particular selected from the group consisting of: Alamethicin, Melittin, Magainin and Dermaseptin,
c) a pore-forming transmembrane protein with beta—barrel transmembrane structure, in particular selected from the group consisting of: Rhodobacter capsulatus porin, Rhodopseudomonas blastica porin, OmpF, PhoE, OmpK36, Omp, Maltoporin, LamB, ScrY, FepA, FhuA, TolC and alpha hemolysine,
d) a transmembrane structure of a pore-forming transmembran protein, e.g. a part, a homolog and/or a part of a homolog of the proteins mentioned in a), b) and c), whereby the transmembrane structure crosses the membrane and forms of a pore in the membrane and
e) a protein that crosses the membrane and forms of a pore in the membrane having a homologous structure as the proteins in accordance with a), b), c) and/or d) whereby homologous structure means a tertiary and/or quaternary structure which forms a pore when assembled in the membrane.

In one embodiment the vesicles of the invention comprises at least one polypeptide with the activity of an outer membrane channel protein.

In one embodiment the vesicles of the invention comprises at least one polypeptide with the activity of an channel protein selected from the group consisting of: porins, preferably OmpF, PhoE, LamB, FepA, Tsx and FhuA and a part thereof and a homologue.

In one embodiment the vesicles of the invention comprises at least one polypeptide with the activity of an channel protein selected from the group consisting of: FhuA, FhuA(delta1-20), FhuA(delta1-40), FhuA(delta1-63), FhuA(delta1-105), FhuA(delta1-160) and a part thereof and a homologue.
In one embodiment the vesicles of the invention comprises at least one polypeptide with the activity of FhuA(delta1-160) protein or a homologue.

In accordance with the invention, a protein or polypeptide has the “activity” of a protein as shown in paragraphs [0021] to [0023] if its expression directly or indirectly leads to a polypeptide, which forms a pore when it is intercalated or assembled in a membrane.

In one embodiment the cross-section of the intercalated channel polypeptide ranges between 1 nm and 30 nm, 2 nm and 20 nm, 2 nm and 10 nm preferably between 2.7 nm and 4.6 nm, between 2.7 nm and 3.8 nm, more preferably between 3.9 nm and 4.0 nm.
In one embodiment the height of the intercalated channel polypeptide ranges between 2 nm and 20 nm, preferably between 4.0 nm and 10.0 nm, between 5.0 nm and 7.0 nm, more preferably between 6.5 nm and 6.9 nm.

In one embodiment the vesicles of the invention comprises at least one polypeptide encoded by a nucleic acid molecule comprising a nucleic acid molecule selected from the group consisting of:

a) nucleic acid molecule encoding of the polypeptide as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 or a fragment thereof, which forms a pore when it is intercalated or assembled in a membrane;
b) nucleic acid molecule comprising of the nucleic acid molecule shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35;
c) nucleic acid molecule whose sequence can be deduced from a polypeptide sequence encoded by a nucleic acid molecule of (a) or (b) as a result of the degeneracy of the genetic code and which forms a pore when it is intercalated or assembled in a membrane;
d) nucleic acid molecule which encodes a polypeptide which has at least 50% identity with the amino acid sequence of the polypeptide encoded by the nucleic acid molecule of (a) to (c) and forming a pore when it is intercalated or assembled in a membrane;
e) nucleic acid molecule which hybridizes with a nucleic acid molecule of (a) to (c) under stringent hybridisation conditions and forming a pore when it is intercalated or assembled in a membrane;
f) nucleic acid molecule which encompasses a nucleic acid molecule which is obtained by amplifying nucleic acid molecules from a cDNA library or a genomic library using the primers as shown in SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85 or SEQ ID NO:86 and forming a pore when it is intercalated or assembled in a membrane;
g) nucleic acid molecule encoding a polypeptide which is isolated with the aid of monoclonal antibodies against a polypeptide encoded by one of the nucleic acid molecules of (a) to (f) and forming a pore when it is intercalated or assembled in a membrane;
and
h) nucleic acid molecule which is obtainable by screening a suitable nucleic acid library under stringent hybridization conditions with a probe comprising one of the sequences of the nucleic acid molecule of (a) to (h) or with a fragment thereof having at least 15 nt, preferably 20 nt, 30 nt, 50 nt, 100 nt, 200 nt or 500 nt of the nucleic acid molecule characterized in (a) to (g) and forming a pore when it is intercalated or assembled in a membrane.

The invention further relates to an isolated nucleic acid molecule comprising a nucleic acid molecule selected from the group consisting of:

a) nucleic acid molecule encoding of the polypeptide as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 or a fragment thereof, which forms a pore when it is intercalated or assembled in a membrane;
b) nucleic acid molecule comprising of the nucleic acid molecule shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35
c) nucleic acid molecule whose sequence can be deduced from a polypeptide sequence encoded by a nucleic acid molecule of (a) or (b) as a result of the degeneracy of the genetic code and which forms a pore when it is intercalated or assembled in a membrane;
d) nucleic acid molecule which encodes a polypeptide which has at least 50% identity with the amino acid sequence of the polypeptide encoded by the nucleic acid molecule of (a) to (c) and forming a pore when it is intercalated or assembled in a membrane;
e) nucleic acid molecule which hybridizes with a nucleic acid molecule of (a) to (c) under stringent hybridisation conditions and forming a pore when it is intercalated or assembled in a membrane;
f) nucleic acid molecule which encompasses a nucleic acid molecule which is obtained by amplifying nucleic acid molecules from a cDNA library or a genomic library using the primers as shown in SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85 or SEQ ID NO:86 and forming a pore when it is intercalated or assembled in a membrane;
g) nucleic acid molecule encoding a polypeptide which is isolated with the aid of monoclonal antibodies against a polypeptide encoded by one of the nucleic acid molecules of (a) to (f) and forming a pore when it is intercalated or assembled in a membrane;
and
h) nucleic acid molecule which is obtainable by screening a suitable nucleic acid library under stringent hybridization conditions with a probe comprising one of the sequences of the nucleic acid molecule of (a) to (h) or with a fragment thereof having at least 15 nt, preferably 20 nt, 30 nt, 50 nt, 100 nt, 200 nt or 500 nt of the nucleic acid molecule characterized in (a) to (g) and forming a pore when it is intercalated or assembled in a membrane,
whereby the nucleic acid molecule distinguishes over the sequence as indicated in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35 by one or more nucleotides.

An “isolated” polynucleotide or nucleic acid molecule is separated from other polynucleotides or nucleic acid molecules, which are present in the natural source of the nucleic acid molecule. An isolated nucleic acid molecule may be a chromosomal fragment of several kb, or preferably, a molecule only comprising the coding region of the gene. Accordingly, an isolated nucleic acid molecule of the invention may comprise chromosomal regions, which are adjacent 5′ and 3′ or further adjacent chromosomal regions, but preferably comprises no such sequences which naturally flank the nucleic acid molecule sequence in the genomic or chromosomal context in the organism from which the nucleic acid molecule originates (for example sequences which are adjacent to the regions encoding the 5′- and 3′-UTRs of the nucleic acid molecule). In various embodiments, the isolated nucleic acid molecule used in the process according to the invention may, for example comprise less than approximately 5 kb, 4 kb, 3 kb, 2 kb, 1 kb, 0.5 kb or 0.1 kb nucleotide sequences which naturally flank the nucleic acid molecule in the genomic DNA of the cell from which the nucleic acid molecule originates.

A nucleic acid molecule encompassing a complete sequence of the nucleic acid molecules used in the invention, or a part thereof may additionally be isolated by polymerase chain reaction, oligonucleotide primers based on this sequence or on parts thereof being used. For example, a nucleic acid molecule comprising the complete sequence or part thereof can be isolated by polymerase chain reaction using oligonucleotide primers which have been generated on the basis of this very sequence. For example, mRNA can be isolated from cells (for example by means of the guanidinium thiocyanate extraction method of Chirgwin et al. (1979) Biochemistry 18:5294-5299) and cDNA can be generated by means of reverse transcriptase (for example Moloney MLV reverse transcriptase, available from Gibco/BRL, Bethesda, Md., or AMV reverse transcriptase, obtainable from Seikagaku America, Inc., St. Petersburg, Fla.).

Synthetic oligonucleotide primers for the amplification, e.g. as shown in SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85 or SEQ ID NO:86 by means of polymerase chain reaction can be generated on the basis of a sequence shown herein.

Moreover, it is possible to identify conserved regions from various organisms by carrying out protein sequence alignments with the polypeptide used in the process of the invention, in particular with sequences of the polypeptide of the invention, from which conserved regions, and in turn, degenerate primers can be derived. Conserved regions are those, which show a very little variation in the amino acid in one particular position of several homologs from different origin.

Degenerated primers can then be utilized by PCR for the amplification of fragments of novel proteins having above-mentioned activity.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as hybridization probe and following standard hybridization techniques under stringent hybridization conditions. In this context, it is possible to use, for example, isolated nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the process of the invention or encoding a protein used in the invention or of the nucleic acid molecule of the invention. Nucleic acid molecules with 30, 50, 100, 250 or more nucleotides may also be used.

The term “homology” means that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalent have the similar immunological characteristic, e.g. comprise similar epitopes.

By “hybridizing” it is meant that such nucleic acid molecules hybridize under conventional hybridization conditions, preferably under stringent conditions such as described by, e.g., Sambrook (Molecular Cloning; A Laboratory Manual, 2nd Edition, Cold Spring Harbor Laboratory Press, Cold Spring Harbor, N.Y. (1989)) or in Current Protocols in Molecular Biology, John Wiley & Sons, N.Y. (1989), 6.3.1-6.3.6.

According to the invention, DNA as well as RNA molecules of the nucleic acid of the invention can be used as probes. Further, as template for the identification of functional homologues Northern blot assays as well as Southern blot assays can be performed. The Northern blot assay advantageously provides further informations about the expressed gene product: e.g. expression pattern, occurance of processing steps, like splicing and capping, etc. The Southern blot assay provides additional information about the chromosomal localization and organization of the gene encoding the nucleic acid molecule of the invention.

A preferred, nonlimiting example of stringent hydridization conditions are hybridizations in 6× sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 58° C., preferably between 45° C. and 50° C. in an aqueous buffer with a concentration of 0.1×0.5×, 1×, 2×, 3×, 4× or 5×SSC (pH 7.2). If organic solvent(s) is/are present in the abovementioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The abovementioned hybridization temperatures are determined for example for a nucleic acid approximately 100 bp (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC: 0.3M sodium citrate, 3M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like i) length of treatment, ii) salt conditions, iii) detergent conditions, iv) competitor DNAs, v) temperature and vi) probe selection can be combined case by case so that not all possibilities can be mentioned herein.

Thus, in a preferred embodiment, Northern blots are prehybridized with Rothi-Hybri-Quick buffer (Roth, Karlsruhe) at 68° C. for 2 h. Hybridzation with radioactive labelled probe is done overnight at 68° C. Subsequent washing steps are performed at 68° C. with 1×SSC.
For Southern blot assays the membrane is prehybridized with Rothi-Hybri-Quick buffer (Roth, Karlsruhe) at 68° C. for 2 h. The hybridzation with radioactive labelled probe is conducted over night at 68° C. Subsequently the hybridization buffer is discarded and the filter shortly washed using 2×SSC; 0.1% SDS. After discarding the washing buffer new 2×SSC; 0.1% SDS buffer is added and incubated at 68° C. for 15 minutes. This washing step is performed twice followed by an additional washing step using 1×SSC; 0.1% SDS at 68° C. for 10 min.

Some examples of conditions for DNA hybridization (Southern blot assays) and wash step are shown hereinbelow:

(1) Hybridization conditions can be selected, for example, from the following conditions:

### a) 4×SSC at 65° C.,

### b) 6×SSC at 45° C.,

c) 6×SSC, 100 mg/ml denatured fragmented fish sperm DNA at 68° C.,
d) 6×SSC, 0.5% SDS, 100 mg/ml denatured salmon sperm DNA at 68° C.,
e) 6×SSC, 0.5% SDS, 100 mg/ml denatured fragmented salmon sperm DNA, 50% formamide at 42° C.,
f) 50% formamide, 4×SSC at 42° C.,
g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
h) 2× or 4×SSC at 50° C. (low-stringency condition), or
i) 30 to 40% formamide, 2× or 4×SSC at 42° C. (low-stringency condition).
(2) Wash steps can be selected, for example, from the following conditions:
a) 0.015 M NaCl/0.0015 M sodium citrate/0.1% SDS at 50° C.

### b) 0.1×SSC at 65° C.

### c) 0.1×SSC, 0.5% SDS at 68° C.

d) 0.1×SSC, 0.5% SDS, 50% formamide at 42° C.

### e) 0.2×SSC, 0.1% SDS at 42° C.

f) 2×SSC at 65° C. (low-stringency condition).

Further, some applications have to be performed at low stringency hybridisation conditions, without any consequences for the specificity of the hybridisation. For example, a Southern blot analysis of total DNA could be probed with a nucleic acid molecule of the present invention and washed at low stringency (55° C. in 2×SSPE, 0.1% SDS). The hybridisation analysis could reveal a simple pattern of only genes encoding polypeptides of the present invention or used in the process of the invention, e.g. having herein-mentioned activity of increasing the fine chemical. A further example of such low-stringent hybridization conditions is 4×SSC at 50° C. or hybridization with 30 to 40% formamide at 42° C. Such molecules comprise those which are fragments, analogues or derivatives of the polypeptide of the invention or used in the process of the invention and differ, for example, by way of amino acid and/or nucleotide deletion(s), insertion(s), substitution (s), addition(s) and/or recombination (s) or any other modification(s) known in the art either alone or in combination from the above-described amino acid sequences or their underlying nucleotide sequence(s). However, it is preferred to use high stringency hybridisation conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, or 30 bp. Preferably are also hybridizations with at least 100 bp or 200, very especially preferably at least 400 bp in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence referred to or hybridizing with the nucleic acid molecule of the invention or used in the process of the invention under stringed conditions, while the maximum size is not critical. In some applications, the maximum size usually is not substantially greater than that required to provide the desired activity and/or function(s) of the original sequence.

Accordingly, the invention relates to nucleic acid molecules encoding a polypeptide having above-mentioned activity, e.g. a channel or pore forming activity when intercalated in a membrane. Such polypeptides differ in amino acid sequence from a sequence contained in the sequences shown SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 yet retain said activity described herein. The nucleic acid molecule can comprise a nucleotide sequence encoding a polypeptide, wherein the polypeptide comprises an amino acid sequence at least about 50% identical to an amino acid sequence shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74. Preferably, the protein encoded by the nucleic acid molecule is at least about 60% identical to the sequence shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 more preferably at least about 70% identical to one of the sequences shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74, even more preferably at least about 80%, 90%, 95% homologous to the sequence shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74, and most preferably at least about 96%, 97%, 98%, or 99% identical to the sequence shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74.

To determine the percentage homology (=identity, herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology” as used in the present context corresponds to amino acid or nucleic acid “identity”. The percentage homology between the two sequences is a function of the number of identical positions shared by the sequences (i.e. % homology=number of identical positions/total number of positions×100). The terms “homology” and “identity” are thus to be considered as synonyms.

For the determination of the percentage homology (=identity) of two or more amino acids or of two or more nucleotide sequences several computer software programs have been developed. The homology of two or more sequences can be calculated with for example the software fasta, which presently has been used in the version fasta 3 (W. R. Pearson and D. J. Lipman (1988), Improved Tools for Biological Sequence Comparison. PNAS 85:2444-2448; W. R. Pearson (1990) Rapid and Sensitive Sequence Comparison with FASTP and FASTA, Methods in Enzymology 183:63-98; W. R. Pearson and D. J. Lipman (1988) Improved Tools for Biological Sequence Comparison. PNAS 85:2444-2448; W. R. Pearson (1990); Rapid and Sensitive Sequence Comparison with FASTP and FASTA Methods in Enzymology 183:63-98). Another useful program for the calculation of homologies of different sequences is the standard blast program, which is included in the Biomax pedant software (Biomax, Munich, Federal Republic of Germany). This leads unfortunately sometimes to suboptimal results since blast does not always include complete sequences of the subject and the querry. Nevertheless as this program is very efficient it can be used for the comparison of a huge number of sequences. The following settings are typically used for such a comparisons of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query define [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx)[Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351-360, 1987, Higgins et al., CABIOS, 5 1989: 151-153) or preferably with the programs Gap and BestFit, which are respectively based on the algorithms of Needleman and Wunsch [J. Mol. Biol. 48; 443-453 (1970)] and Smith and Waterman [Adv. Appl. Math. 2; 482-489 (1981)]. Both programs are part of the GCG software-package [Genetics Computer Group, 575 Science Drive, Madison, Wis., USA 53711 (1991); Altschul et al. (1997) Nucleic Acids Res. 25:3389 et seq.]. Therefore preferably the calculations to determine the perentages of sequence homology are done with the program Gap over the whole range of the sequences. The following standard adjustments for the comparison of nucleic acid sequences were used: gap weight: 50, length weight: 3, average match: 10.000, average mismatch: 0.000.

For example a sequence, which has 80% homology with sequence SEQ ID NO:1 at the nucleic acid level is understood as meaning a sequence which, upon comparison with the sequence SEQ ID NO:1 by the above Gap program algorithm with the above parameter set, has a 80% homology.

Homology between two polypeptides is understood as meaning the identity of the amino acid sequence over in each case the entire sequence length which is calculated by comparison with the aid of the program algorithm GAP (Wisconsin Package Version 10.0, University of Wisconsin, Genetics Computer Group (GCG), Madison, USA), setting the following parameters:

| | | | |
|---|---|---|---|
| Gap weight: | 8 | Length weight: | 2 |
| Average match: | 2,912 | Average mismatch: | −2,003 |

For example a sequence which has a 80% homology with sequence SEQ ID NO:36 at the protein level is understood as meaning a sequence which, upon comparison with the sequence SEQ ID NO:36 by the above program algorithm with the above parameter set, has a 80% homology.

Functional equivalents derived from one of the polypeptides as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 according to the invention and are distinguished by essentially the same properties as the polypeptide as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74.

Functional equivalents derived from the nucleic acid sequence as shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35 according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 according to the invention and encode polypeptides having essentially the same properties as the polypeptide as shown in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74.

Essentially the same properties” of a functional equivalent is above all understood as meaning that the functional equivalent has above mentioned activity, e.g a channel or pore forming activity when intercalated in a membrane.

A nucleic acid molecule encoding an homologous to a protein sequence of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73 or SEQ ID NO:74 can be created by introducing one or more nucleotide substitutions, additions or deletions into a nucleotide sequence of the nucleic acid molecule of the present invention, in particular of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35 such that one or more amino acid substitutions, additions or deletions are introduced into the encoded protein. Mutations can be introduced into the encoding sequences of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34 or SEQ ID NO:35 by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophane), beta-branched side chains (e.g., threonine, valine, isoleucine) and aromatic side chains (e.g., tyrosine, phenylalanine, tryptophane, histidine).

Thus, a predicted nonessential amino acid residue in a polypeptide of the invention or a polypeptide used in the process of the invention is preferably replaced with another amino acid residue from the same family. Alternatively, in another embodiment, mutations can be introduced randomly along all or part of a coding sequence of a nucleic acid molecule of the invention or used in the process of the invention, such as by saturation mutagenesis, and the resultant mutants can be screened for activity described herein to identify mutants that retain or even have increased above mentioned activity, e.g. conferring an increase in content of the fine chemical.

Homologues of the nucleic acid sequences used, with the sequence shown in sid, comprise also allelic variants with at least approximately 30%, 35%, 40% or 45% homology, by preference at least approximately 50%, 60% or 70%, more preferably at least approximately 90%, 91%, 92%, 93%, 94% or 95% and even more preferably at least approximately 96%, 97%, 98%, 99% or more homology with one of the nucleotide sequences shown or the abovementioned derived nucleic acid sequences or their homologues, derivatives or analogues or parts of these. Allelic variants encompass in particular functional variants which can be obtained by deletion, insertion or substitution of nucleotides from the sequences shown or from the derived nucleic acid sequences.

In one embodiment the vesicles of the invention comprises at least one pore forming polypeptide selected from the group as disclosed in EP 1559790 A1, paragraphs [0022] to [0031].

In one embodiment of the invention the transmembrane channel protein is labeled.

In one embodiment of the invention the transmembrane channel protein is labeled with an labeling agent selected from the group of:

amino-, hydroxyl-, carboxyl- and sulfhydryl-, diazo group labeling agents.
In one embodiment of the invention the transmembrane channel protein is labeled with an amino-group labeling agent.
According to the invention an amino-group labeling agent is a compound which reacts and covalently binds to a (free) amino-group of an amino acid from a polypeptide.

The amino-group labeling agent is a compound which comprises a further functional group.

In one embodiment the functional group reacts with a compound and is elongated. The product of this reaction is selected from the group consisting of:
ether, ester, thioether, thioester, dithioether, disulfide, amine, amide, bond to gold or coordinated to complexed metals, especially Rh, Ru, Fe, Ni, Cu, Zn.
In one embodiment the functional group reacts with a compound and is shortened, e.g. a part of the labeling agent is split off. The product of this reaction is selected from the group consisting of:
alcohol, organic acid, thiol, sulfide, amine.

In one embodiment the compound reacting with the functional group is selected from the group consisting of:

alcohol, organic acid, thiol, sulfide, alkaline solution, oxidation agent and reduction agent.
In one embodiment the compound reacting with the functional group is a reduction agent selected from the group consisting of:
Disulfid bond reducing agents such as 1,4-Dithio-DL-threitol (DTT), mercaptoethanol, metals such as Zn in acid solution.
In one embodiment the compound reacting with the functional group is diazo bond breaking reagent such as sodium borate at pH 9

In one embodiment of the invention the transmembrane channel protein is labeled with an amino-group labeling agent selected from the group consisting of: isothiocyanates, isocyanates, acyl azides, NHS esters, sulfonylchlorides, aldehydes and glyoxals, epoxides and oxiranes, imidoesters, carbodiimides, anhydrides, 3-(2-Pyridyldithio)propionic acid N-hydroxysuccinimide ester and biotin disulfide N-hydroxysuccinimide ester.

In one embodiment of the invention the transmembrane channel protein is labelled with pyridyl- and biotinyl-labels at six lysine residues.
In one embodiment of the invention the transmembrane channel protein is labeled with an thiol-group labeling agent selected from the group consisting of:
Thiol-disulfide exchange reagents, arylting agents, acryloyl derivatives, aziridines, maleimides, haloacetyl and alkylhalide derivatives. In one embodiment of the invention the transmembrane channel protein is labeled with a hydroxyl-group labeling agent selected from the group consisting of:
Epoxides and oxiranes, carbonyldiimidazole, Disuccinimidyl carbonate, N-hydroxysuccinimidyl chloroformate, alkyl halogens and isocyantes.

The invention further provides a process for the production of the vesicles according to the invention comprising the following steps:

a) adding a transmembrane channel protein to a solution or dispersion of a labeling agent and,
b) adding the labeled transmembrane channel protein to a solution or dispersion of molecules forming a vesicle
c) forming the vesicle whereby the labeled transmembrane channel protein is incorporated into the membrane of the vesicle.

In one embodiment of the invention the transmembrane channel protein cloned, expressed, extracted and purified according to the well known methods disclosed in e.g., Sambrook (Molecular Cloning; A Laboratory Manual, 2nd Edition, Cold Spring Harbor Laboratory Press, Cold Spring Harbor, N.Y. (1989)).

In an other embodiment the transmembrane channel protein is cloned, expressed, extracted and purified according to Nallani et al., pages 51 to 53, chapter 2.1, 2.2 or according to EP 1559790 A1, paragraphs [0053] to [0058].

In one embodiment of the invention the vesicle is formed by a method selected from the group consisting of:

direct dispersion method, ethanol method, ink jet, extrusion and film hydration method.

In an other embodiment the vesicle is formed by a method according to Nallani et al., page 53, chapter 2.3 or according to EP 1559790 A1, paragraphs [0059] to [0061].

The invention further provides a process for the production of the vesicles according to the invention comprising the following steps:

a) adding a transmembrane channel protein to a solution or dispersion of a labeling agent and,
b) adding the labeled transmembrane channel protein to a solution or dispersion of molecules forming a vesicle
c) adding the solution or dispersion from step b) to a solution or dispersion containing the substance to be charged within the vesicle,
d) forming the vesicle whereby the labeled transmembrane channel protein is incorporated into the membrane of the vesicle and the substance is encapsulated into the vesicle.

In one embodiment of the invention the vesicle is formed by a method selected from the group consisting of:

direct dispersion method, ethanol method, ink jet, extrusion and film hydration method. In an other embodiment the vesicle is formed by a method according to Nallani et al., page 53, chapter 2.3 or according to EP 1559790 A1, paragraphs [0059] to [0061].

The invention further provides a method for triggering the transmembrane transport adding to the dispersion containing the vesicle according to the invention a compound which opens the transmembrane channel protein by splitting off at least a part of the labeling agent in a chemical reaction.

In one embodiment the chemical reaction is selected from the group consisting of: reduction, oxidation, substitution or thermal- pH- or light-triggered instability of the labeling agent.

The triggering according to the invention allows the controlled start and/or stop or reversible start/stop, preferably start, of the passage of compounds through a vesicle with a selective permeable membrane.

It also allows a selection of the compound which traverse the membrane by combination of the channel forming polypeptide with different labeling agents. A further selection is possible by choice of the compound reacting with the labeling agent.

The trigger does not only control the moment of the transmembrane transport but additionally the velocity of the transport, e.g. the amount of translocated compound in a certain time.

The vesicle of the invention is used for controlled enzyme catalyzed reactions. Enzymes are encapsulated into the vesicles in order to protect them from shear forces, proteases or organic solvents present in the biocatalysis media while the transport of substrate and respective product is mediated by the membrane protein.

In one embodiment the enzyme is selected from the group consisting of: beta-lactamase, hydrolase, lipase, oxidase, peroxidase and dehydrogenase.
The enzymes are encapsulated by a method according to Nallani et al., page 55, chapter 2.7 and Onaca et al., page 799, chapter 2.5.1 to 2.5.3.

The vesicle of the invention is used for controlled selected recovery and release of charged compounds. Charged polymeric compounds are encapsulated into the vesicles in order to protect them from shear forces or organic solvents present in the media while the transport of compounds with opposite charge is mediated by the membrane protein.

In one embodiment the charged polymeric compounds is selected from the group consisting of:
polycationic molecukes like polyhistidine, or polyanionic molecules like DNA, RNA or anionic polysaccharides.
The charged polymeric compounds are encapsulated by a method according to Nallani et al., page 54, chapter 2.6 and Onaca et al., page 799, chapter 2.5.4.

In order to capture single strained DNA, like primers, nanophosphor DNA conjugates are encapsulated in the vesicles of the invention.

The nanophosphor DNA conjugates are encapsulated by a method according to Onaca et al., page 799, chapter 2.5.5.
The same conjugates can be used to captures RNA, especially short RNA fragments such as siRNA.

### EXAMPLES

All chemicals used were of analytical reagent grade or higher quality and purchased from Sigma-Aldrich Chemie (Taufkirchen, Germany) and Applichem (Darmstadt, Germany) if not stated otherwise. FhuA Δ1-160 variant was expressed, extracted and purified as previously described (Nallani) until homogeneity using E. coli B^{E} strain BL 21 (DE3) omp8 (F⁻ hsdS_{B} (r_{B}⁻ m_{B}⁻) gal ompT dcm (DE3) ΔlamB ompF::Tn5 ΔompA ΔompC) {Prilipov, 1998}. Protein concentrations were determined using the standard BCA kit (Pierce Chemical Cc, Rockford, USA).

FhuA Δ1-160 Labeling and Nanocompartment Formation

FhuA Δ1-160 (50 μL, 4 μM) was added drop-wise to a DMSO (100 μL) solution containing 3-(2-Pyridyldithio)propionic acid N-hydroxysuccinimide ester (76.8 mM) or (2-[Biotinamido]ethylamido)-3,3′-dithiodipropionic acid N-hydroxysuccinimide ester (8.2 mM) and stirred (3000 rpm, 1 h; RCT basic IKAMAG, IKA-Werke GmbH, Staufen, Germany). The latter solution was used for formation of nanocompartments loaded with calcein (50 mM) according to a previously reported Ethanol method {Nallani} without further work-up.

ABA (PMOXA-PDMS-PMOXA) triblock copolymer (50 mg; Mw ˜20000 g/mol) was dissolved in ethanol (250 μl; 99.8%) and stirred for 30 min. The clear solution was added drop-wise into Tris-KCl buffer (5 ml; 10 mM Tris, 100 mM KCl, pH 7.4) containing calcein (50 mM) and stirred (3000 rpm; ambient temperature; 3-4 h). Nanocompartments loaded with calcein (50 mM), harboring FhuA Δ1-160 (0.13 μM final concentration) as well as amino group labeled FhuA Δ1-160 (0.13 μM final concentration) were prepared using the Ethanol method {Nallani} with the concentrations and volumes described. Nanocompartments formed by self-assembly were subsequently extruded (6 times; 0.22 μm Milex filter (Millipore Corporation, Bedford, Mass., USA)) to form uniform spherically shaped nanocompartments

Nanocompartments were purified by gel filtration using Sepharose 43 (Sigma-Aldrich) in Tris-KCl buffer (5 ml; 10 mM Tris, 100 mM KCl, pH 7.4) as previously described {Graff, 2002}. Average diameters of nanocompartments were routinely determined using a Zeta-Sizer (Zeta-Sitar Nano Series; Malvern, Worcestershire, United Kingdom).

Calcein Release Assay with Synthosomes

An excitation wavelength of 480 nm and an emission wavelength of 520 nm were used for all calcein release measurements. Fast kinetics were recorded for 15 minutes (kinetic interval 1 μs) using a Cary Eclipse Fluorescence Spectrophotometer (Varian, Inc. Corporate Headquarters, Palo Alto, USA). For measurements up to 120 minutes a Saphire Fluorescence Spectrophotometer (Team Trading AG, Mannedorf/Zurich, Switzerland) was employed (kinetic interval 60 s). In fast kinetic measurements a purified nanocompartment or Synthosome suspension (500 μl; Tris-KCl buffer (5 ml; 10 mM Tris, 100 mM KCl, pH 7.4) was supplemented with DTT (10 μl, 1 M), mixed gently by pipetting (Eppendorf, Hamburg, Germany), and used in each experiment Five hundred-μl suspension were rapidly transferred into quartz cuvettes (Hellma GmbH&Co. KG, Müllheim, Germany) for recording calcein release kinetics.

For long time measurements 200 μl of chromatographically purified nanocompartment or Synthosome suspension were supplemented with DTT (10 μl, 1 M) in a mircotiter plate (Flat-Bottom, Black, 96 well, Greiner Bio-One, Frikenhausen, Germany), mixed with a pipette (Eppendorf, Hamburg, Germany), and used in each experiment. Integrity of nanocompartments and Synthosomes was determined by comparing size distribution and intensity in dynamic light scattering experiments using Zeta-Sizer (Zeta-Sizer Nano Series; Malvern, Worcestershire, United Kingdom).

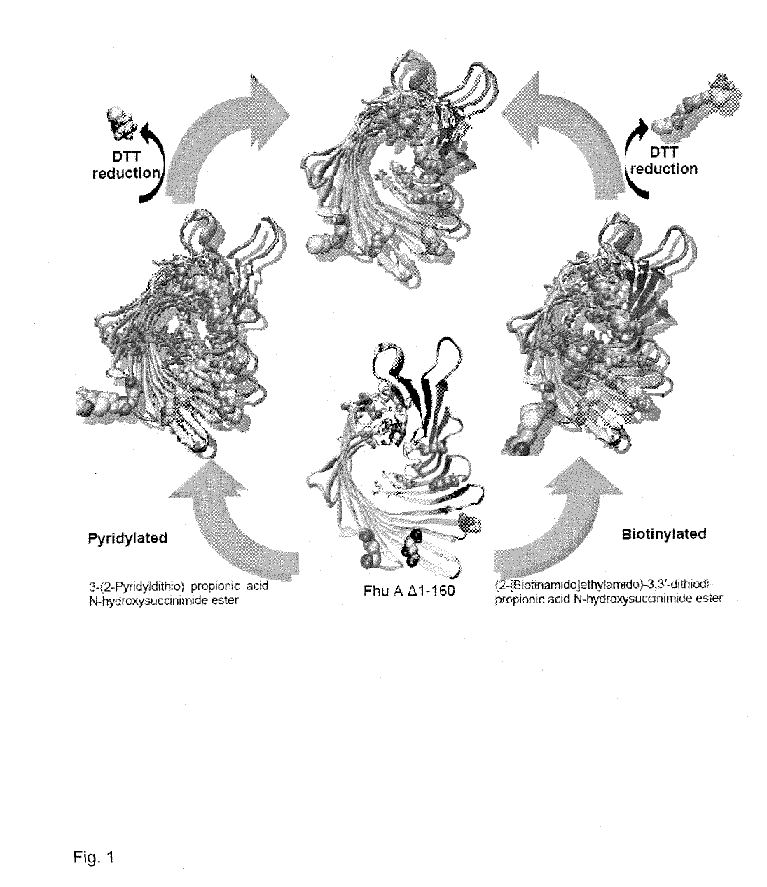

FIG. 1 shows the reaction schemes of six chemically modified lysines (167, 344, 364, 537, 556, 586; FhuA Δ1-160) with a pyridyl- (left) and a biotinyl-label (right). Upon disulfide bond reduction with DTT, a 3-Thio-propionic amide group remains for both labels at these six lysines residues of FhuA Δ1-160 (FIG. 1; upper part). The top view on the FhuA Δ1-160 channel in FIG. 1 provides an impression how the pyridyl- and biotinyl-label restrict translocation after lysine modification. A densely packed β-barrel can especially be observed for the sterically more demanding biotinyl-label. FIG. 1 impressively indicates how a tunnel might opens up at the “left” transmembrane channel part after releasing the biotin- and pyridyl-labels by DTT addition

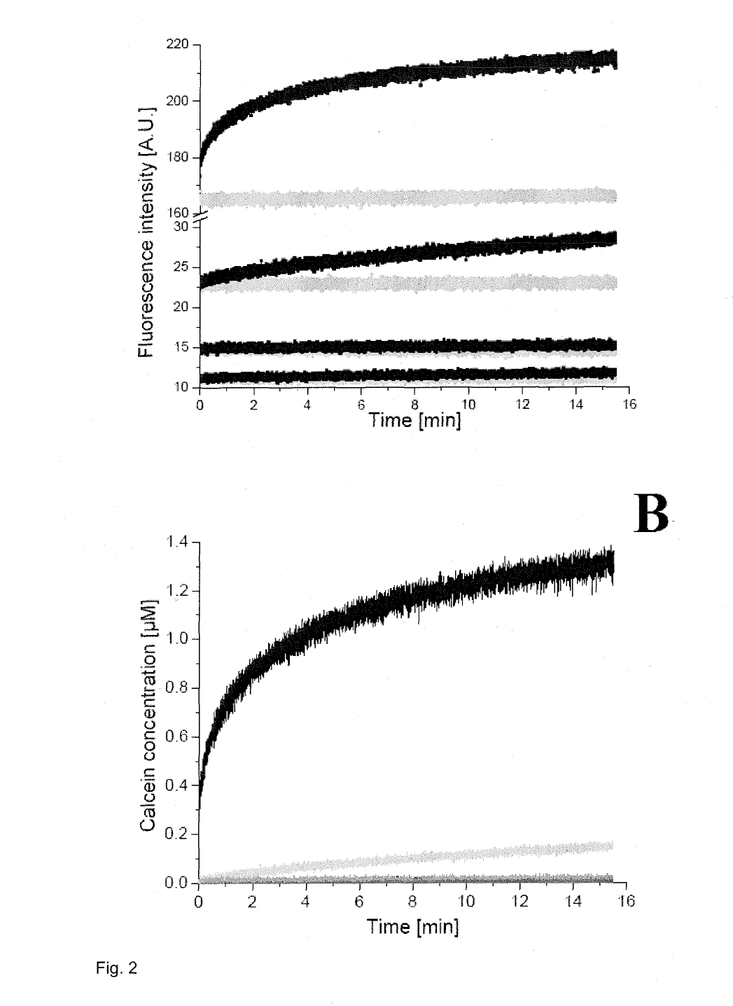

FIG. 2 shows calcein release kinetics in arbitrary units (A) and absolute calcein concentrations (B) of Synthosomes before and after adding the reduction trigger DTT. In absence of a FhuA Δ1-160 transmembrane protein there is no detectable calcein release before and after DTT addition (FIG. 2; data set-11 AU; 1). In case of the unlabeled FhuA Δ1-160 one could expect that calcein translocates through FhuA Δ1-160 as previously shown (Nallani) and is therefore lost during Synthosome purification (FIG. 2; data set-15AU; 2). For the biotin-labeled FhuA Δ1-160 a linear release kinetic can be observed after DTT reduction with an initial rate of 0.59 min⁻¹ for the first 2 minutes. A ˜30-fold faster (first 2 minutes) and exponential initial calcein release can be observed for the statically less demanding Pyridyl-label, which leads upon reduction to the same 3-Thio-propionic amide-label FhuA Δ1-160 (FIG. 1). The strong size dependence of the initial release kinetics indicates a stronger non-covalent binding of the biotin-label inside the FhuA Δ1-160 channel. Interestingly the release kinetics reach in both instances nearly identical rates after ˜six minutes which remain nearly constant for 2 h (data not shown). These findings further support a stronger non-covalent binding of biotin inside the channel preventing its rapid release.

For a better comparison of calcein release kinetics in Table 1 we have used the following empirical formula:

$$C = - P 1 exp ( - \frac{t}{P 2} ) + P 3 ,$$

in which P1, P2, P3 are empirical parameters that are calculated from experimental calcein kinetics (FIG. 2). Parameter P1 depends on the difference in calcein concentration inside the nanocompartments and outside the nanocompartments in the bulk solution. The significant higher values for the Pyridyl-label FhuA Δ1-160 channel (FIG. 2; Tabel 1; 28.5 Pyridyl- vs. 7.4 Biotinyl-) can be attributed to a FhuA Δ1-160 limited diffusion since the employed samples were after purification analyzed by a Zeta-Sizes and in quantity normalized by elution peaks. Parameter P2 represents the time constant of the calcein release process describing the efflux from nanocompartment sample through the FhuA Δ1-160 channel protein. Pyridinyl-labeled FhuA Δ1-160 shows upon unblocking a four times faster time constant (3.99 min) than the Biotinyl-labeled FhuA Δ1-160 (time constant of 13.88 min). P2 depends on the number of FhuA 1-160 molecules per nanocompartment, the FhuA Δ1-160 channel properties (size, charge, dynamics, chemical labeling), and DTT concentrations. Apart from the labeled amino groups all factors were identical; differences are therefore directly linked to employed labeling reagents. Parameter P3 describes the background fluorescence of the nanocompartment systems. The significant higher background values for the Pyridyl-labeled FhuA Δ1-160 channel (FIG. 2) can be attributed to a slow release of calcein during storage. The Pyridyl-labeled FhuA Δ1-160 suspension shows a calcein fluorescence build up after storage overnight in contrast to the Biotin-label FhuA Δ1-160 suspension. In case of long time fluorescence recordings after 120 minutes a difference of 32.89% for Pyridyl-labeled FhuA Δ1-160 higher than Biotinyl-labeled FhuA Δ1-160 in release rate was calculated.

TABLE 1
Empirical formula
$C = - P 1 exp ( - \frac{t}{P 2} ) + P 3$
used to compare calcein release kinetics of
pyridylated and biotinylated FhuA Δ1-160 Synthosomes by determining
P1, P2 and P3 from recorded release kinetics.

| | | | P3 |
|---|---|---|---|
| Sample | | P2 | [A.U.] |
| Calcein concentrations in “()” | P1 | [min] | (μM) |
| 1. Nanocompartments | — | — | 11.58 |
| | | | (0.03) |
| 2. Nanocompartments-FhuA Δ1-160 | — | — | 14.53 |
| | | | (0.039) |
| 3. Nanocompartments with FhuA Δ1-160 | 28.53 | 3.99 | 165.72 |
| blocked with pyridyl derivative | | | (0.06) |
| 4. Nanocompartments with FhuA Δ1-160 | 7.24 | 13.88 | 23.76 |
| blocked with biotinyl derivative | | | (0.46) |

1. A vesicle comprising at least one transmembrane transport trigger system.

2. The vesicle according to claim 1, wherein the trigger system comprises a transmembrane efflux and/or influx trigger system.

3. The vesicle according to claim 1 comprising at least one transmembrane channel protein.

4. The vesicle according to claim 3, wherein the transmembrane channel protein comprises at least one outer membrane channel protein.

5. The vesicle according to claim 3, wherein the at least one transmembrane channel protein is selected from the group consisting of: porins, OmpF, PhoE, LamB, FepA, Tsx, and FhuA or a part or a homologue thereof.

6. The vesicle according to claim 3, wherein the at least one transmembrane channel protein is labeled.

7. The vesicle according to claim 6, wherein the at least one transmembrane channel protein is labeled with an amino-group labeling agent.

8. The vesicle according to claim 7, wherein the amino-group labeling agent is selected from the group consisting of: 3-(2-Pyridyldithio)propionic acid N-hydroxysuccinimide ester and biotin disulfide N-hydroxysuccinimide ester.

9. The vesicle according to claim 1, whereby the vesicle is selected from the group consisting of: liposome, polymersome and synthosome.

10. The vesicle according to claim 1 comprising an encapsulating membrane.

11. The vesicle of claim 10 wherein the encapsulating membrane comprises block copolymers.

12. A process for the production of the vesicle of claim 1 comprising:
a) adding a transmembrane channel protein to a solution or dispersion of a labeling agent;
b) adding the labeled transmembrane channel protein obtained in a) to a solution or dispersion of molecules forming a vesicle; and
c) forming a vesicle whereby the labeled transmembrane channel protein is incorporated into the membrane of the vesicle.

13. The process according to claim 12 further comprising:
adding the solution or dispersion from step b) to a solution or dispersion containing the substance to be charged within the vesicle, and
forming the vesicle whereby the labeled transmembrane channel protein is incorporated into the membrane of the vesicle and the substance is encapsulated into the vesicle.

14. A method for triggering transmembrane transport comprising adding to a dispersion containing the vesicle of claim 7 a substance which opens the transmembrane channel protein by splitting off at least a part of the labeling agent in a chemical reaction.

15. The method according to claim 14, wherein the chemical reaction is selected from the group consisting of: reduction, oxidation and substitution.

16. The vesicle according to claim 3, where the protein comprises a pore forming protein.

17. The vesicle of claim 5, wherein the at least one channel protein is selected from the group consisting of: FhuA, FhuA(delta1-20), FhuA(delta1-40), FhuA(delta1-63), FhuA(delta1-105), and FhuA(delta1-160) or a part or a homologue thereof.

18. The method of claim 15, wherein the chemical reaction is a reduction with a reduction agent selected from the group consisting of a disulfid bond reducing agent, 1,4-Dithio-DL-threitol (DTT), mercaptoethanol, a metal, and Zn in acid solution.

19. The vesicle of claim 6, wherein the at least one transmembrane channel protein is labelled with pyridyl- and biotinyl-labels at six lysine residues.
